(19) United States
(12) Patent Application Publication
(10) Pub. No.: US 2026/0144471 A1
(43) Pub. Date: May 28, 2026

(54) NANO-ELECTRODE AND METHOD OF MANUFACTURING THE SAME

(71) Applicants: Georgia Tech Research Corporation, Atlanta, GA (US); Florida Institute for Human & Machine Cognition, Inc., Pensacola, FL (US)

(72) Inventors: Vladimir Tsukruk, Atlanta, GA (US); Jinyoung Kim, Atlanta, GA (US); Sehyun Park, Atlanta, GA (US); Jisoo Jeon, Atlanta, GA (US); Dong-hee Kang, Atlanta, GA (US); Gwendolyn M. Bryan, Pensacola, FL (US); Timothy J. Broderick, Pensacola, FL (US); Morley O. Stone, Pensacola, FL (US)

(21) Appl. No.: 19/402,474

(22) Filed: Nov. 26, 2025

Related U.S. Application Data
(60) Provisional application No. 63/725,728, filed on Nov. 27, 2024.

(51) Int. Cl. A61B 5/259; A61B 5/28; A61B 5/296; C23C 16/56

(52) U.S. Cl. CPC A61B 5/259; A61B 5/28; A61B 5/296; C23C 16/56; A61B 2562/0285; A61B 2562/125

(57) ABSTRACT

A nano-electrode includes a hydrophilic conductive layer and a hydrophobic non-conductive layer. The hydrophilic conductive layer includes a first side surface and a second side surface opposite from the first side surface. The hydrophobic non-conductive layer includes a first side surface and a second side surface opposite from the first side surface. The hydrophobic non-conductive layer is configured to cover the first side surface of the hydrophilic conductive layer. The second side surface of the hydrophilic conductive layer is configured to adhere to a skin surface.

### CROSS-REFERENCE TO RELATED APPLICATIONS

This application claims priority to U.S. Provisional Patent Application No. 63/725,728, filed Nov. 27, 2024, entitled “NANO-ELECTRODE AND METHOD OF MANUFACTURING THE SAME,” the entirety of which is incorporated herein by reference.

### GOVERNMENT FUNDING

This invention was made with government support under FA8650-22-2-5410 awarded by the U.S. Air Force Research Lab, and 2202907 awarded by the National Science Foundation. The government has certain rights in the invention.”

### BACKGROUND

This disclosure relates generally to nano-electrodes, and more particularly to an ultrathin skin-conformal nano-electrode configured for high-fidelity electrophysiological signal monitoring in dynamic and wet conditions and a method for manufacturing the same.

Skin-mounted electronics involve the development of wearable sensors that adhere directly to the skin for various applications such as healthcare monitoring, fitness tracking, and human-machine interaction. Notably, epidermal electrodes placed on the skin enable the accurate extraction of electrophysiological signals such as electrocardiogram (ECG), electromyogram (EMG), electrooculogram, and electroencephalogram. These electrodes offer continuous monitoring of biopotentials, aiding in the diagnosis and treatment of cardiac, neurological, and muscular disorders, as well as assessing skin impedance and hydration levels. Specifically, accurate detection, long-term usability of skin biopotentials, resilience to motion artifacts, and long-term environmental/water stability, are of great importance for real-time human health monitoring.

Conventional electrophysiological electrodes, such as gel-type and dry contact electrodes, face several critical limitations including high motion artifacts, high skin interfacial impedance, low signal-to-noise ratio (SNR), and short-term usage under harsh environmental conditions. For example, conventional gel-type electrodes are short-lived and prone to motion artifacts and skin irritation because the Ag/AgCl gel electrolyte is vulnerable to environmental factors and sweat absorption. These factors disrupt the stability of the gel, which leads to poor, unstable contact to the skin and decreased signal quality over a long time of use. In addition, dry contact electrodes such as thin metal, conductive polymer, and composites rely on direct contact with the skin surface. However, this contact is affected by air gaps, the loss of contact, and tribological charging, which severely compromise electrophysiological signals, particularly in dynamic environments. Therefore, the present disclosure recognizes a growing need and interest in improved electrophysiological electrodes such as ultrathin electrodes, e-tattoos, and smart adhesives for resilient direct mounting on the skin.

It has been suggested that nanoscale-ultrathin flexible electrodes (thickness below 500 nm)?provide close contact on dynamic curvilinear surfaces, attributed to low bending stiffness for firmly attaching to the skin. However, achieving a seamless, highly adherent, and void-free contact with skin remains a challenge, as air gaps tend to form at the skin-electrode interface. This issue is more pronounced in micrometer-thick electrodes, which exhibit greater mismatch in interfacial mechanics, leading to motion artifacts during intense shearing and bending at the electrode-skin interface.

Therefore, the present disclosure addresses the need to design a highly conformal contact, water-assisted capillary-driven flow, which can be utilized to adhere the electrode to the skin, ensuring intimate contact, reduced noise, and high-fidelity electrophysiological signal acquisition. In particular, the inventors have identified that the control of hydrophilicity on the surface of the electrode, as well as conformal contact, provides mechanical and electrical stability leading to resilience to low-motion artifacts during intense motions. It prevents water penetration at the skin-electrode interface, thereby enabling the acquisition of reliable signals for extended times under diverse conditions, including an underwater environment. In view of the present disclosure, tuning surface hydrophilicity of the skin-electrode interface, improvements to electrical contact and adhesion in wet physiological environments can be achieved, and enhanced mechanical/electrical stability and reliable electrophysiological signal acquisition over an extended period of time in dynamic and water-rich environments can be made possible.

### SUMMARY

The present disclosure may provide a nano-electrode that comprises a hydrophilic conductive layer having orthogonal first and second side surfaces, and a hydrophobic non-conductive layer configured to cover the first side surface of the hydrophilic conductive layer, wherein the second side surface of the hydrophilic conductive layer is configured to adhere to skin.

In certain examples, a thickness of the hydrophobic non-conductive layer is about 300 nm; a thickness of the 50 nm thickness of hydrophilic conductive layer is about 50 nm; the hydrophilic conductive layer comprises a MXene nanosheet; and/or the hydrophobic non-conductive layer comprises parylene. As will be understood by one skilled in the art, MXenes are named after their chemical formula, M₁₊₁XₙT_{y}, where M is an early transition metal (most commonly Ti, Zr, Hf, V, or Nb) and X is carbon or nitrogen. These are a class of two-dimensional inorganic compounds that consist of atomically thin layers of transition metal carbides, nitrides, or carbonitrides. The structure of MXene sheets includes n+1 M layers that are intercalated with the n X layers and covered with the T groups.

In accordance with aspects of the present disclosure, a method of fabricating a nano-electrode is presented. In some embodiments, the method of fabricating a nano-electrode includes coating a glass substrate with a sacrificial layer, depositing a parylene on the sacrificial layer, coating the parylene with an MXene solution, annealing a combination of the sacrificial layer, the parylene, and the MXene solution to form the nano-electrode, and removing the sacrificial layer.

In some embodiments, the parylene is a parylene film, and the depositing parylene on the sacrificial layer includes chemical vapor deposition (CVD) of a parylene film onto the sacrificial layer. In some embodiments, the depositing parylene on the sacrificial layer includes treating the parylene film with an O₂ plasma to prepare the parylene as a hydrophilic surface.

In some embodiments, the method includes coating the parylene with a poly-L-lysine (PLL) solution. In some embodiments, the coating of the parylene with the poly-L-lysine (PLL) solution is performed by spin-coating. In some embodiments, the annealing of the combination of the sacrificial layer, parylene and the MXene is performed at a temperature of about 110° C. for a period of about 10 minutes. In some embodiments, the nano-electrode formed has a thickness of about 350 nm. In some embodiments, the PLL layer formed has a thickness of about 3 nm.

In some embodiments, the nano-electrode may be transferred and attached to a skin surface. In some embodiments, the nano-electrode formed on the glass substrate is placed in water. In some embodiments the nano-electrode detaches from the glass substrate as the sacrificial layer dissolves in the water. The hydrophobicity of the parylene layer and the hydrophilicity of the MXene layer cause the nano-electrode to float in the water, with the parylene layer facing upwards toward the air, and the MXene layer facing downwards in contact with the water.

In some embodiments, a method of applying a nano-electrode to a skin surface includes floating the nano-electrode in water, submerging the skin surface in the water under the nano-electrode, stretching the skin surface, lifting the skin surface being stretched until the nano-electrode mounts to the skin surface, releasing the skin surface, and drying the nano-electrode mounted on the skin surface.

In accordance with aspects of the present disclosure, a nano-electrode includes a hydrophilic conductive layer and a hydrophobic non-conductive layer. In some embodiments, the hydrophilic conductive layer includes a first side surface and a second side surface opposite from the first side surface. In some embodiments, the hydrophobic non-conductive layer includes a first side surface and a second side surface opposite from the first side surface.

In some embodiments, the hydrophobic non-conductive layer is configured to cover the first side surface of the hydrophilic conductive layer. In some embodiments, the second side surface of the hydrophilic conductive layer is configured to contact skin or a skin surface. In some embodiments, the second side surface of the hydrophilic conductive layer is configured to adhere to the skin or the skin surface.

In some embodiments, a thickness of the hydrophobic non-conductive layer is about 200 nm to 400 nm. In some embodiments, a thickness of the hydrophobic non-conductive layer is about 250 nm to 350 nm. In some embodiments, a thickness of the hydrophobic non-conductive layer is about 275 nm to 325 nm. In some embodiments, a thickness of the hydrophobic non-conductive layer is about 300 nm. In some embodiments, the hydrophobic non-conductive layer includes parylene. In some embodiments, the hydrophobic non-conductive layer includes crosslinked chemical vapor deposition (CVD) fabricated parylene nanofilm.

In some embodiments, a thickness of the hydrophilic conductive layer is about 40 nm to 60 nm. In some embodiments, a thickness of the hydrophilic conductive layer is about 45 nm to 55 nm. In some embodiments, a thickness of the hydrophilic conductive layer is about 50 nm. In some embodiments, the hydrophilic conductive layer includes at least one of an MXene, an MXene layer, an MXene sheet, an MXene nanosheet, and an MXene film. In some embodiments, the hydrophilic conductive layer includes a conductive layer of a 2D MXene nanosheet phase (Ti₃C₂Tₓ).

In some embodiments, the nano-electrode further comprises an adhesive layer configured to attach the hydrophilic conductive layer and the hydrophobic non-conductive layer. In some embodiments, the adhesive layer contacts the first side surface of the hydrophilic conductive layer and the adhesive layer contacts the second side surface and the hydrophobic non-conductive layer. In some embodiments, the adhesive layer includes poly-L-lysine (PLL).

In some embodiments, a nano-electrode includes a hydrophilic layer with a MXene material and a hydrophobic layer with parylene. The hydrophilic layer has a first side surface and a second side surface opposite the first side surface, and the hydrophobic layer covers the first side surface of the hydrophilic layer.

In some embodiments, the second side surface of the hydrophilic layer is configured to self-adhere to the skin surface. In some embodiments, a thickness of the hydrophobic layer is about 250 nm to 350 nm. In some embodiments, a thickness of the hydrophobic layer is about 300 nm. In some embodiments, a thickness of the hydrophilic layer is about 40 nm to 60 nm. In some embodiments, a thickness of the hydrophilic layer is about 50 nm.

This summary is not intended to identify essential features of the claimed subject matter, nor is it intended for use in determining the scope of the claimed subject matter. It is to be understood that both the foregoing general description and the following detailed description are exemplary and are intended to provide an overview or framework to understand the nature and character of the disclosure.

### BRIEF DESCRIPTION OF THE DRAWINGS

The following detailed description of aspects of the disclosure will be better understood when read in conjunction with the appended drawings. It should be understood, however, that the disclosure is not limited to the precise arrangements and instrumentalities shown. In the drawings:

FIG. 1A shows a schematic illustrating a fabrication process of a skin-conformal nano-electrode in accordance with an embodiment of the present disclosure;

FIG. 1B shows a schematic illustrating a transfer process of nano-electrodes onto human skin for conformal contact in accordance with an embodiment of the present disclosure;

FIG. 1C shows a perspective cross-sectional view showing skin-conformal nano-electrodes in accordance with an embodiment of the present disclosure;

FIG. 1D shows a schematic illustrating water-resistant skin-conformal nanoelectrodes in accordance with an embodiment of the present disclosure;

FIG. 1E is a chart showing determination of parylene thickness required for conformal contact as a function of nano-electrode thickness and the work of adhesion of human skin;

FIG. 1F shows images of a transfer process of the nano-electrodes being applied onto human skin in accordance with aspects of the present disclosure (scale bar in lower right has a length of 2 cm);

FIG. 1G is a chart showing zeta potential of aqueous MXene and 0.1 wt % PPL solutions, in accordance with aspects of the present disclosure;

FIG. 2A shows a perspective view of skin-conformal nano-electrodes of the present disclosure applied onto artificial skin in accordance with an embodiment of the present disclosure;

FIG. 2B shows enlarged views of profilometry height profiles with different parylene thicknesses (bare skin, 300 nm, 1 μm, and 3 μm parylenes) in accordance with an embodiment of the present disclosure;

FIG. 2C shows charts of a roughness analysis in linear depth (line: 5 mm) with different parylene thicknesses (bare skin, 300 nm, 1 μm, and 3 μm parylenes) in accordance with an embodiment of the present disclosure;

FIG. 2D shows a chart of 3D areal surface (area: 0.5 mm×3.5 mm) with different parylene thicknesses (bare skin, 300 nm, 1 μm, and 3 μm parylenes) in accordance with an embodiment of the present disclosure;

FIG. 2E shows cross-sectional SEM images with different electrode thickness (300 nm top and 1 μm bottom) in accordance with an embodiment of the present disclosure;

FIG. 2F shows a perspective view of a setup for peeling strength measurement with an area (1.5 cm×4.0 cm) in accordance with an embodiment of the present disclosure;

FIG. 2G shows a chart of results for peel strength test of nano-electrodes with different thicknesses delaminated from artificial skin (peeling rate of 50 mm/min) in accordance with an embodiment of the present disclosure;

FIG. 2H shows a chart of optical transmittance of a skin-conformal nano-electrode depending on different MXene concentrations in accordance with an embodiment of the present disclosure;

FIG. 2I shows a schematic design of the multilayered skin-conformal nano-electrodes in accordance with aspects of the present disclosure;

FIG. 2J shows images of the skin-conformal nano-electrodes with different concentrations of MXene solutions (5-25 mg/mL) in accordance with aspects of the present disclosure (scale bar in lower right has a length of 1 cm);

FIG. 2K shows an image of a commercial watch with a PPG sensor operable to collect data to perform BPM monitoring while the PPG sensor is placed over the skin-conformal nano-electrodes in accordance with aspects of the present disclosure (scale bar in lower right has a length of 1 cm);

FIG. 3A shows an enlarged image of skin-conformal nano-electrodes applied onto human skin (scale bar in lower right has a length of 2 cm);

FIG. 3B shows a further enlarged view of FIG. 3A detailing a boundary between the human skin and the skin-conformal nano electrodes (scale bar in lower right has a length of 50 μm);

FIG. 3C shows images of the skin-conformal nano-electrodes in an undeformed state, and in states where it is subjected to spread, pinch and torsion (scale bar in lower right has a length of 500 μm);

FIG. 3D shows an SEM image of the skin-conformal nano-electrodes with dome-like patterned array (15 μm diameter and 2 μm pitch) (scale bar in lower right has a length of 20 μm);

FIG. 3E shows side views of contact angles of water on the MXene (top pane) and the parylene surfaces (bottom pane);

FIG. 3F shows images of cold water applied to the skin-conformal nano-electrodes (left pane) and after removal of the skin-conformal nano-electrodes with soapy water (right pane) (scale bar in lower right has a length of 2 cm);

FIG. 3G shows images of skin-conformal nano-electrodes applied to human skin with varying hair density in accordance with aspects of the present disclosure (scale bar in lower right has a length of 10 mm);

FIG. 3H shows a chart with peel strength results for the skin-conformal nano-electrodes with different thicknesses delaminating from artificial skin (peeling rate of 50 mm/min);

FIG. 3I shows an optical image of the conformal contact using 100 nm-thick parylene electrodes on the skin in accordance with aspects of the present disclosure (scale bar in lower right has a length of 1 mm);

FIG. 3J shows SEM images of the 0.5 and 1-μm-thick parylene electrodes coating the dome-like patterned array in accordance with aspects of the present disclosure (scale bar in lower right has a length of 10 μm);

FIG. 3K shows images of the skin-conformal nano-electrodes on the skin after a 5-kilometer run (scale bar in lower right has a length of 1 cm);

FIG. 3L shows a chart of water vapor transmission (scale of 0-4 g/cm²) through the skin-conformal nano-electrodes with different parylene thicknesses under relative humidity of 20±10% at 35° C. (with data plots arranged from top to bottom: open, nano-electrode, medical adhesive, 1-μm parylenes, 3-μm parylenes, and closed) in accordance with aspects of the present disclosure;

FIG. 3M shows a chart of water vapor transmission (scale of 0-0.5 g/cm²) through the skin-conformal nano-electrodes with different parylene thicknesses under relative humidity of 20±10% at 35° C. (with data plots arranged from top to bottom: open, nano-electrode, medical adhesive, 1-μm parylenes, 3-μm parylenes, and closed) in accordance with aspects of the present disclosure;

FIG. 3N shows a chart with evaluation of cell compatibility for biocompatibility of skin-conformal nano-electrodes in accordance with aspects of the present disclosure;

FIG. 4A shows a chart of interfacial impedances of nano-electrodes (lower plot of data points), gel-electrodes (center plot of data points, and thick-electrodes (upper plot of data points);

FIG. 4B shows a chart of long-term (4 hour) impedance stability of the skin-conformal nano-electrodes in accordance with an embodiment of the present disclosure;

FIG. 4C shows a chart of underwater (30 minute) impedance of nano-electrodes and gel-electrodes at 20 Hz in accordance with an embodiment of the present disclosure;

FIG. 4D shows a chart of relative resistance changes of nano-electrodes and thick-electrodes as a function of forward and backward bending in accordance with an embodiment of the present disclosure;

FIG. 4E shows a chart of sheet resistance to transmittance at 550 nm for MXene suspensions with different MXene concentrations in accordance with an embodiment of the present disclosure;

FIG. 4F shows an AFM analysis for thickness of the MXene layer from scratch profiling (inset pane) in accordance with an embodiment of the present disclosure (scale bar in lower right has a length of 5 μm);

FIG. 4G shows a chart of skin interfacial underwater impedance at different times for nano-electrodes;

FIG. 4H shows a chart of skin interfacial underwater impedance at different times for gel-electrodes;

FIG. 4I shows images of skin-conformal nano-electrodes attached to an inner wrist and then subjected to forward and backward bending in accordance with an embodiment of the present disclosure (scale bar in lower right has a length of 2 cm);

FIG. 4J shows a chart on the effects of repeated bending deformations on the resistance change of the skin-conformal nano-electrodes (curvature radius of 5 cm and 5000 cycles);

FIG. 4K shows a chart of roughness analysis of 3D areal surface (area of 9 mm×12 mm);

FIG. 4L shows a chart on effects of compressive bending deformations (radius of curvature 5 cm and 1000 bending cycles) on change of profilometry height profiles for nano-electrodes (top) and thick parylene electrodes (bottom) progressing from bare skin (left), before compressive bending (middle), and after compressive bending) (scale bar in lower right has a length of 2 mm);

FIG. 4M shows effects of tensile stress (30% stress) on the change in resistance to skin-conformal nano-electrode and the resulting optical images of stretched nano-electrodes on silicone elastomer substrate (Ecoflex 00-30) (scale bar in lower right has a length of 1 mm);

FIG. 4N shows a chart of the relative resistance variations on the nano-electrodes up to 30% strain;

FIG. 4O shows a chart of repeated tensile stress on the resistance change of the nano-electrodes over 5000 cycles;

FIG. 4P shows charts of long-term (10-days) electrical stability of the skin-conformal nano-electrodes (size 3 cm×2 cm) that were affixed to artificial skin, with conductive ink on medical tape used to connect to both ends of the electrodes;

FIG. 5A shows a schematic of an ECG measurement setup where two electrodes were attached to inner wrists of right and left arms, and the other one was attached to the back of the left-hand as the ground electrode in accordance with an embodiment of the present disclosure;

FIG. 5B shows a chart of ECG signals measured by nano-electrodes (upper plot of data points), gel-electrodes (center plot of data points), and thick-electrodes (lower plot of data points) at rest condition in accordance with an embodiment of the present disclosure;

FIG. 5C shows a schematic of repetitive normal (left pane) and forward bending (right pane) motions with the skin-conformal nano-electrodes attached to the wrist in accordance with an embodiment of the present disclosure;

FIG. 5D shows a chart of ECG signals measured by nano-electrodes (upper plot of data points), gel-electrodes (center plot of data points), and thick-electrodes (lower plot of data points) during repetitive bending motions in accordance with an embodiment of the present disclosure;

FIG. 5E shows a chart of ECG signals of nano-electrodes (upper plot of data points), gel-electrodes (center plot of data points), and thick-electrodes (lower plot of data points) during repetitive bending motions in accordance with an embodiment of the present disclosure;

FIG. 5F shows a schematic of underwater ECG monitoring with skin-conformal nano-electrodes in accordance with an embodiment of the present disclosure;

FIG. 5G shows a chart of long-term ECG monitoring of nano-electrodes (upper plot of data points), gel-electrodes (center plot of data points), and thick-electrodes (lower plot of data points) in accordance with an embodiment of the present disclosure;

FIG. 5H shows a chart of continuous ECG monitoring of nano-electrodes (upper plot of data points), gel-electrodes (center plot of data points), and thick-electrodes (lower plot of data points) during activities such as rest (normal), forward bending, backward bending, rest (underwater), and bending (underwater) in accordance with an embodiment of the present disclosure;

FIG. 5I shows a chart of T/R ratio and TP derivations of measured by nano-electrodes (upper bar of each group), gel-electrodes (center bar of each group), and thick-electrodes (lower bar of each group) in accordance with an embodiment of the present disclosure;

FIG. 5J shows a comparison chart of PQRST waveforms of the SNR analysis of nano-electrodes (left plot), gel-electrodes (center plot), and thick electrodes (right plot);

FIG. 5K shows a short-time Fourier transform spectrogram up to 50 Hz of the nano-electrode (scale bar in lower right correspond to 1 second);

FIG. 5L shows a top schematic view of interface design between the nano-electrode and the interconnected electrical circuit for wrist bending test in accordance with aspects of the present disclosure;

FIG. 5M shows a schematic side view of interface design between the nano-electrode and the interconnected electrical circuit wrist bending test in accordance with aspects of the present disclosure;

FIG. 5N shows a chart of spectral density estimates for nano-electrodes, gel-electrodes, and thick electrodes in a static state;

FIG. 5O shows a chart of spectral density estimates for nano-electrodes, gel-electrodes, and thick electrodes under forward bending;

FIGS. 5P, 5Q, and 5R show enlarged plots of the data summarized in FIG. 5H showing continuous ECG monitoring with nano-electrodes (upper plot of data points), gel-electrodes (center plot of data points), and thick-electrodes (lower plot of data points);

FIG. 6A shows a schematic of an EMG measurement setup in which two electrodes were attached to the left forearm and the other one was attached on the back of the right-hand as a ground electrode in accordance with an embodiment of the present disclosure;

FIG. 6B shows a chart of EMG signals measured by nano-electrodes (upper plot of data points), gel-electrodes (center plot of data points), and thick-electrodes (lower plot of data points) while gripping a stress ball, tennis ball, and baseball in accordance with an embodiment of the present disclosure;

FIG. 6C shows a chart of a SNR analysis of nano-electrodes (upper plot of data points), gel-electrodes (center plot of data points), and thick-electrodes (lower plot of data points) while gripping a stress ball, tennis ball, and baseball in accordance with an embodiment of the present disclosure;

FIG. 6D shows a chart of continuous EMG monitoring nano-electrodes (left bar of each group), gel-electrodes (center bar of each group), and thick-electrodes (right bar of each group) in the air and underwater during finger and motion movements in accordance with an embodiment of the present disclosure;

FIG. 6E shows a chart of baseline noise and SNR analysis of bending motion EMG signals in air (solid) and underwater (stripe) in accordance with an embodiment of the present disclosure;

FIG. 6F shows a chart of consistent EMG signals under various deformations (pinch, torsion, spread, and touch) in accordance with an embodiment of the present disclosure;

FIG. 7A shows a chart of continuous ECG monitoring in different environments (normal, in a sauna, at rest, and in a pool) in accordance with an embodiment of the present disclosure;

FIG. 7B shows a chart of HR monitoring based on R-peak detection in accordance with an embodiment of the present disclosure;

FIG. 7C shows a chart of standard deviation of RR intervals in different environments (normal, in a sauna, at rest, and in a pool) in accordance with an embodiment of the present disclosure;

FIG. 7D shows a chart of Poincare plot in different environments (normal, in a sauna, at rest, and in a pool) in accordance with an embodiment of the present disclosure;

FIG. 7E shows block diagrams of Pan-Tompkins algorithm for R-peak detection from raw ECG signals in accordance with an embodiment of the present disclosure;

FIG. 8A shows an image of skin-conformal nano-electrode attached to a tibialis anterior muscle at a distance of 5 mm in accordance with an embodiment of the present disclosure;

FIG. 8B shows an image of a subject walking on a treadmill for monitoring of ECG and EMG signals simultaneously in accordance with an embodiment of the present disclosure (scale bar in lower right has a length of 20 cm);

FIG. 8C shows an enlarged image of the EMG monitoring of FIG. 8B;

FIG. 8D shows a chart of continuous EMG monitoring of the tibialis anterior muscle using nano-electrodes during walking at different speeds (1.0 m/s, 1.4 m/s, and 1.8 m/s) in accordance with an embodiment of the present disclosure;

FIG. 8E shows a chart of continuous HR monitoring of the tibialis anterior muscle using nano-electrodes during walking at different speeds (1.0 m/s, 1.4 m/s, and 1.8 m/s) in accordance with an embodiment of the present disclosure;

FIG. 8F shows a chart of SNR analysis of nano-electrodes (left bar of each group), gel-electrodes (right bar of each group) in accordance with an embodiment of the present disclosure;

FIG. 8G shows a chart of baseline noise analysis of nano-electrode over 2 days in accordance with an embodiment of the present disclosure;

FIG. 8H shows images of gait cycle events matched with the corresponding natural tibialis anterior EMG patterns in accordance with an embodiment of the present disclosure (scale bar in lower right has a length of 20 cm);

FIG. 8I shows a chart of intensity of the envelope profile of nano-electrodes (upper plot of data points) and gel-electrodes (lower plot of data points) over time in accordance with an embodiment of the present disclosure;

FIG. 8J shows a chart of intensity of the envelope profile of nano-electrodes (upper plot of data points) and gel-electrodes (lower plot of data points) over the percentage of gait cycle in accordance with an embodiment of the present disclosure;

FIG. 8K shows a top schematic view of interface design between the nano-electrode and the interconnected electrical circuit for human gait motion tests in accordance with aspects of the present disclosure;

FIG. 8L shows a side schematic view of interface design between the nano-electrode and the interconnected electrical circuit for human gait motion test in accordance with aspects of the present disclosure;

FIG. 8M shows a chart of continuous EMG monitoring of tibialis anterior muscle using gel-electrodes during walking at three different speeds (1.0 m/s, 1.4 m/s, and 1.8 m/s);

FIG. 8N shows a comparison of the EMG signals of the nano-electrodes (upper plot of data points) and gel-electrodes (lower plot of data points) in tibialis anterior muscle at 1.0 m/s.

FIG. 8O shows images after long-term use of nano-electrodes and gel-electrodes on skin for 1 day and 6 hours;

FIG. 8P show charts of a signal processing protocol of linear envelop for monitoring tibialis anterior where data is processed through a high pass filter, followed by full wave rectification, and then low pass filtering to produce a linear envelope of data movement in accordance with aspects of the present disclosure;

FIG. 8Q shows an image of AFM analysis to estimate the thickness of the skin-conformal nano-electrodes (scale bar in lower right has a length of 2 μm);

FIG. 8R shows a plot of the thickness of the skin-conformal nano-electrodes;

FIG. 8S shows an image of AFM analysis to estimate the thickness of the PLL layer (scale bar in lower right has a length of 4 μm);

FIG. 8T shows a plot of the thickness of the PLL layer; and

FIG. 9 is a schematic illustrating a skin-conformal nano-electrode in accordance with an embodiment of the present disclosure.

### DETAILED DESCRIPTION

It is to be understood that the figures and descriptions of the present disclosure may have been simplified to illustrate elements that are relevant for a clear understanding of the present disclosure, while eliminating, for purposes of clarity, other elements found in a typical wearable assistance device or typical method of using a wearable assistance device. Those of ordinary skill in the art will recognize that other elements may be desirable and/or required in order to implement the present disclosure. However, because such elements are well known in the art, and because they do not facilitate a better understanding of the present disclosure, a discussion of such elements is not provided herein. It is also to be understood that the drawings included herewith only provide diagrammatic representations of the presently preferred structures of the present disclosure and that structures falling within the scope of the present disclosure may include structures different than those shown in the drawings. Reference will now be made to the drawings wherein like structures are provided with like reference designations.

Before explaining at least one embodiment in detail, it should be understood that the inventive concepts set forth herein are not limited in their application to the construction details or component arrangements set forth in the following description or illustrated in the drawings. It should also be understood that the phraseology and terminology employed herein are merely for descriptive purposes and should not be considered limiting.

It should further be understood that any one of the described features may be used separately or in combination with other features. Other invented devices, systems, methods, features, and advantages will be or become apparent to one with skill in the art upon examining the drawings and the detailed description herein. It is intended that all such additional devices, systems, methods, features, and advantages be protected by the accompanying claims.

It will be apparent to those skilled in the art having the benefit of the teachings presented in the foregoing descriptions and the associated drawings that modifications, combinations, sub-combinations, and variations can be made without departing from the spirit or scope of this disclosure. Likewise, the various examples described may be used individually or in combination with other examples. Those skilled in the art will appreciate various combinations of examples not specifically described or illustrated herein that are still within the scope of this disclosure. In this respect, it is to be understood that the disclosure is not limited to the specific examples set forth and the examples of the disclosure are intended to be illustrative, not limiting. Other inventive devices, systems, methods, features, and advantages will be or become apparent to one skill in the art upon examining the drawings and the detailed description herein. It is intended that all such additional devices, systems, methods, features, and advantages be protected by the accompanying claims.

As used in this specification and the appended claims, the singular forms “a”, “an” and “the” include plural referents, unless the context clearly dictates otherwise. Similarly, the adjective “another,” when used to introduce an element, is intended to mean one or more elements. The terms “comprising,” “including,” “having” and similar terms are intended to be inclusive such that there may be additional elements other than the listed elements.

As used herein, the term “about” when used in connection with a referenced numeric indication means the referenced numeric indication plus or minus up to 10 percent of that referenced numeric indication. For example, “about 100” means from 90 to 110.

In a similar manner, the term “substantially” when used in connection with, for example, a geometric relationship, a numerical value, and/or a range is intended to convey that the geometric relationship (or the structures described thereby), the number, and/or the range so defined is nominally the recited geometric relationship, number, and/or range. For example, two structures described herein as being “substantially parallel” is intended to convey that, although a parallel geometric relationship is desirable, some non-parallelism can occur in a “substantially parallel” arrangement. By way of another example, a structure defining a volume that is “substantially 1 millimeter (mm) in height” is intended to convey that, while the recited height is desirable, some tolerances can occur when the measurement of the height is “substantially” the recited height (e.g., 1 mm). Such tolerances can result from manufacturing tolerances, measurement tolerances, and/or other practical considerations (such as, for example, minute imperfections, age of a structure so defined, a pressure or a force exerted within a system, and/or the like). As described above, a suitable tolerance can be, for example, ±10% of the stated geometric construction, numerical value, and/or range. Furthermore, although a numerical value modified by the term “substantially” can allow for and/or otherwise encompass a tolerance of the stated numerical value, it is not intended to exclude the exact numerical value stated.

The present disclosure relates to wearable electrodes with high conformability to the skin to allow for a comfortable, second skin-like wearing experience and record high-quality electrophysiological signals over extended periods, including in challenging environments (such as underwater). The present disclosure addresses the known critical limitations of wearable electronics including proper skin adhesion, excessive motion artifacts, and long-term maintaining of signals under various external conditions. In particular, achieving a conformal skin-electrode interface without air voids provides mechanical and electrical stability and prevents water penetration. The nano-electrode of the present disclosure is configured to be a nanoscale skin-conformal ultrathin electrode that enables continuous resilient electrophysiological signals monitoring with low-motion artifacts and high water-resistance. As disclosed herein, the skin-electrode interface may include a 350 nm flexible nano-electrode with dual hydrophilicity that can integrate, for example, a hydrophilic MXene conductor and a hydrophobic cross-linked parylene layer (e.g. around 300 nm), which can ensure highly conformal contact and long-term stable physical adherence to skin, including under wet conditions. This ultrathin design facilitates high physical adhesion to skin and low skin interfacial impedance for continuous, reliable monitoring of electrocardiogram (ECG), and electromyogram (EMG) signals with a greatly increased signal-to-noise ratio under real-life conditions as compared to commercial gel electrodes.

For example, the nano-electrode of the present disclosure allows for recording of high-quality ECG signals, which allows for the analysis of heart rate and its variability across diverse in-field testing conditions. The nano-electrode also allows for concurrent EMG and ECG monitoring during activities, such as treadmill walking, achieving stable, long-term signal acquisition, particularly in monitoring demanding human activity.

As will be appreciated by one skilled in the art in view of the present disclosure, skin-mounted sensory electronics require durable wearable sensors that adhere directly to the skin for various applications such as healthcare monitoring, fitness tracking, and human-machine interaction. Notably, epidermal electrodes placed on the skin may enable the accurate extraction of electrophysiological signals such as electrocardiogram (ECG), electromyogram (EMG), electrooculogram, and electroencephalogram. These electrodes offer continuous monitoring of biopotentials, aiding in the diagnosis and treatment of cardiac, neurological, and muscular disorders, as well as assessing skin impedance and hydration levels. Specifically, accurate detection, resilience to motion artifacts, and long-term environmental/water stability are great concerns for real-time health monitoring.

It will be appreciated that ultrathin flexible electrodes may provide close contact on dynamic curvilinear surfaces due to low bending stiffness for firmly attaching to the skin. However, achieving a seamless, highly adherent, and void-free contact with skin prior to the present disclosure remained challenging, as air gaps tended to form at the skin-electrode interface. This issue is more pronounced in micrometer-thick electrodes, which exhibit greater mismatch in interfacial mechanics, leading to motion artifacts during intense shearing and bending at the electrode-skin interface.

In particular, the control of hydrophilicity on the surface was previously relatively unexplored, though refining this aspect could prevent water penetration at the skin-electrode interface. Tuning surface hydrophilicity can significantly improve electrical contact and adhesion in wet physiological environments, highlighting the critical role of interfacial wetting engineering for water management in bioelectronic systems. Such strategies can further provide enhanced mechanical/electrical stability and reliable electrophysiological signal acquisition for extended times even in dynamic and water-rich environments. These challenges, and the solutions for overcoming these challenges as identified in the present disclosure, will be explored in greater detail below.

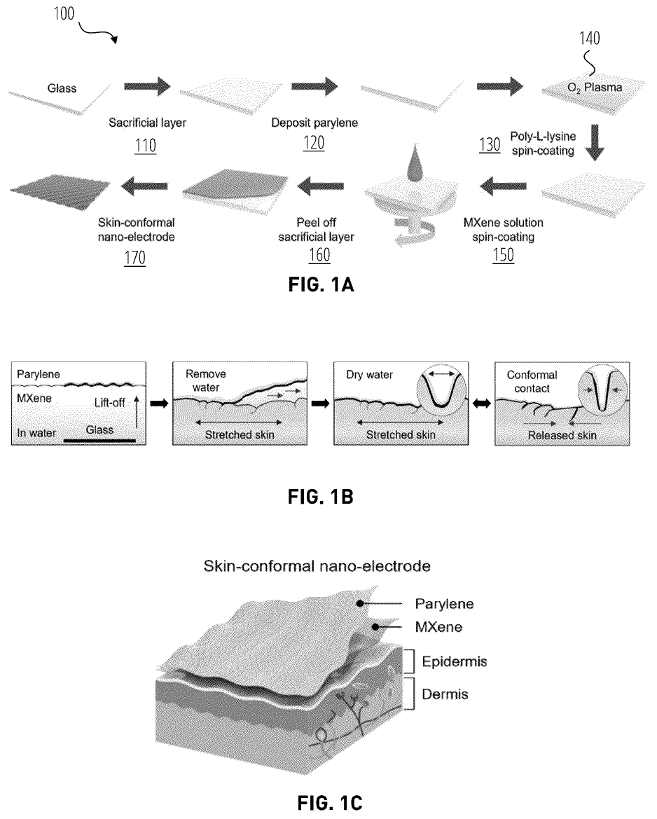

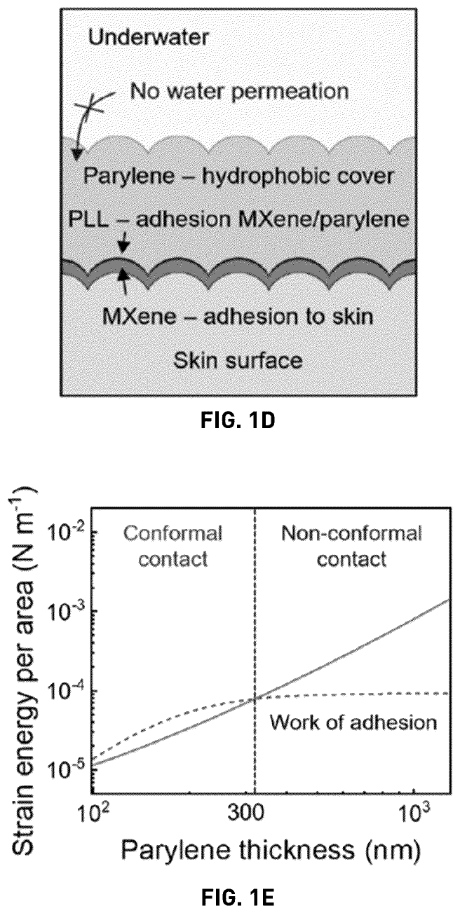

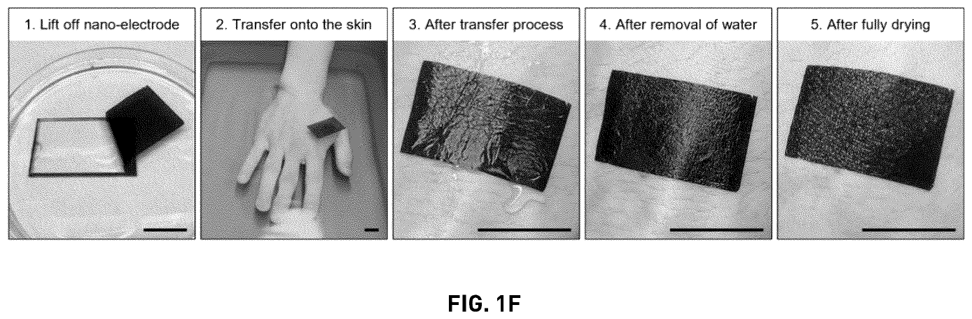

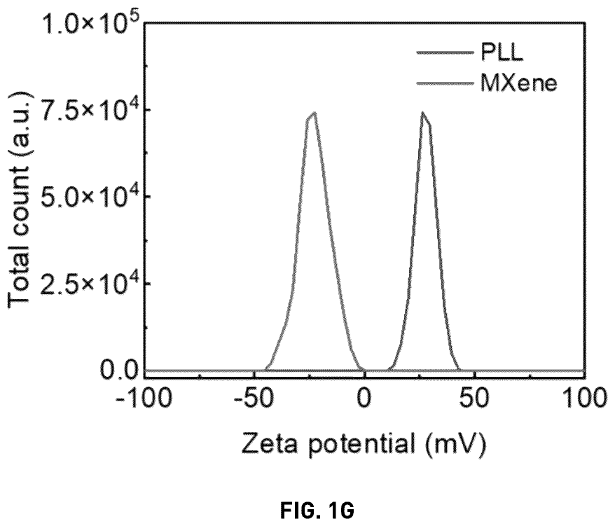

The present disclosure provides a skin-conformal nano-electrode with hydrophilic and hydrophobic sides comprising a highly conductive layer of a 2D MXene phase (Ti₃C₂Tₓ), a poly-L-lysine (PLL) adhesive layer, and an ultrathin cross-linked CVD-fabricated parylene nanofilm (see FIGS. 1A-1C). The transfer process onto human skin, using water-assisted capillary-driven flow, reinforced the high conformability of the skin-electrode interface without air voids, tight physical contact, and high skin adherence. This ultrathin hydrophobic-hydrophilic tri-layered design facilitates the resistance to low-motion artifacts and continuous monitoring such of ECG and EMG signals under challenging external conditions and long sustainable performance in air and underwater unachievable by current thick gel and dry electrodes.

In some embodiments, the ultrathin hydrophobic-hydrophilic tri-layered design of the present disclosure ensured reliable skin interfacial impedance over extended periods (4 hours) and in underwater conditions. In comparison to commercial gel and thick (1-μm-thick parylene) electrodes, the skin-conformal nano-electrodes of the present disclosure demonstrated exceptional ECG and EMG signal quality, characterized by manifold (up to 3.0 times) increase in signal-to-noise ratio and dramatically reduced noise and showed low-motion artifacts, sustainability, and wet environment resistance. Moreover, the inventors demonstrated reliable EMG signal monitoring during various actions, such as gripping balls and finger/motion movements. For proof of concept, inventors used the skin-conformal nano-electrodes to monitor ECG signals, enabling the determination of heart rate (HR) and heart rate variability (HRV) both in extreme environments (e.g., sauna and pool), which was previously unachievable with previously existing sensors. Additionally, the inventors successfully demonstrated the concurrent monitoring of EMG and ECG signals for high-quality muscle activity during a walking cycle and long term (up to 30 hours) stability.

Fabrication and Device Design. In some embodiments, the skin-conformal nano-electrode of the present disclosure can be manufactured using a spin-coating and sacrificial layer-etching process, as illustrated in FIG. 1A. Briefly, first a sacrificial layer (e.g. Micro-90) is coated or fabricated on a cleaned glass substrate followed by parylene chemical vapor deposition and spin coating with the PLL adhesive molecular layer for attaching a nanoscale conducting layer of staggered 2D flakes of MXene. In some embodiments, a concentrated MXene solution (25 mg/mL) can be spin-coated on the PLL adhesive molecular layer and annealed the resulting multilayered film at 110° C. for 10 minutes to remove the residual solvent and adhere layers.

In some embodiments, to achieve enhanced skin interfacial contact and long-term stability (e.g., impedance to low-motion artifact and water environment during electrophysiological signal monitoring), the rolling transfer process of the skin-conformal nano-electrodes onto human skin was implemented (see FIGS. 1B and 1F). The as-fabricated MXene/parylene electrodes were placed in deionized (DI) water to dissolve the sacrificial layer and release the MXene/parylene electrodes in a free standing state. Different hydrophilicities of the parylene (air) and MXene (water) interfaces make the MXene/parylene electrodes float on the water without forming any crumples. After removing the residual water and allowing it to dry completely, the MXene/parylene electrodes can be conformally attached to the pre-stretched skin, leading to high skin-electrode contact, stability, and durability when the skin is released.

The MXene layer of the nano-electrode of the present disclosure acts as a conductive layer helping to perform not only high-level electrophysiological signal detection but also strong hydrophilic and polar interactions with skin and the PLL layer. This material design yields a hydrophilic surface and high adhesion between negatively charged MXene nanosheets and the positively charged amine functional groups of PLL (zeta potential of −22.6 and +26.3 mV, respectively)(see FIG. 1G). In addition, the strong and flexible hydrophobic parylene layer provides stability and integrity of the sensory film, and chemical stability. The hydrophilic side surface (also referred to as a MXene side surface) faces the skin surface, while the hydrophobic surface (also referred to as a parylene side surface) faces outward in FIG. 1C. The hydrophobic layer covers the MXene, namely the first side surface thereof that is opposite the second side surface. This dual hydrophilicity design of the skin-conformal nanoelectrodes prevents direct water penetration at the skin-electrode interface, enabling water-resistant behavior (see FIG. 1d). In some embodiments, the nano-electrodes described herein maintain the contours of the skin, as they are thinner than the spacing and thickness of the human skin surface.

To determine the optimal electrode thickness (which may also be referred to as a critical thickness) for conformal contact with the skin, the inventors calculated the bending-induced strain energy per area and work of adhesion. The inventors utilized an analytical model for interfacial mechanics (see FIG. 1E). The variables in this model include the modulus of MXene and parylene, as well as the roughness of human skin.

Firm conformal contact is achieved when the work of adhesion of the electrode exceeds the bending-induced strain energy. In accordance with aspects of the present disclosure, the electrode in this model comprises two layers: an MXene layer (layer 1), and a parylene layer (layer 2). Based on the optimized thickness of the MXene layer as 50 nm, for example, the critical thickness of the parylene layer can be determined, where conformal contact occurs. The bending-induced strain energy (U_{bending-strain}) per area of the electrode is:

$$\frac{{U}_{bending - strain}}{A} = \frac{{E}_{eff} {t}^{3}}{24 {R}^{2}} \qquad ( 1 )$$

$${E}_{eff} = {\sum }_{i = 1}^{N} \frac{{E}_{i} {t}_{i}}{{t}_{total}} \qquad ( 2 )$$

where U_{bending-strain} is bending-induced strain energy, A is an area of the device, E_{eff} is the effective elastic modulus of the electrode, and E_{eff} is calculated as a composite of N layers, t is the total thickness of the electrode, R is the radius of curvature, E, is the Young's modulus of i^{th} layer, tᵢ is the thickness of i^{th} layer. R was considered to be approximately 1 mm for skin.

In addition, to obtain the work of adhesion on soft human skin as a function of the parylene thickness, the skin surface was assumed as a sinusoidal model, which is described as:

$$y ( x ) = \frac{{h}_{rough}}{2} ( 1 + cos \frac{2 π x}{{λ}_{rough}} ) \qquad ( 3 )$$

where y(x) is skin roughness, h_{rough} is roughness amplitude, and λ_{rough} is wavelength.

Conformal contact induces exact match between the electrode and skin surface. Therefore, electrode displacement (w(x)) and skin displacement (uz(x)) are given as:

$$w ( x ) = \frac{h}{2} ( 1 + cos \frac{2 π x}{{λ}_{rough}} ) \qquad ( 4 )$$

$${u}_{z} ( x ) = y - w = \frac{{h}_{rough} - h}{2} ( 1 + cos \frac{2 π x}{{λ}_{rough}} ) \qquad ( 5 )$$

The maximum deflection of the electrode is h:

$$h = \frac{{E}_{skin} {h}_{rough}}{\frac{16 {π}^{3} EI}{{λ}_{rough}^{3}} + {E}_{skin}} \qquad ( 6 )$$

Conformal contact conditions are defined by calculating interfacial contact energy (U_{conformal}) by:

$${U}_{conformal} = {U}_{bending} + {U}_{skin} + {U}_{adhesion} \qquad ( 7 )$$

where electrode bending energy, skin elastic energy, and contact adhesion energy are denoted as U_{bending}, Uₛₖᵢₙ, and U_{adhesion}, respectively.

Bending energy is calculated as:

$${U}_{bending} = \frac{1}{{λ}_{rough}} {\int }_{0}^{{λ}_{rough}} \frac{{EI ( {w}^{″} )}^{2}}{2} dx = \frac{{π}^{4} {EIh}^{2}}{{λ}_{rough}^{4}} \qquad ( 8 )$$

The MXene and parylene are a layered structure where the effective bending stiffness (EI) is represented as:

$${EI}_{total} = {EI}_{MXene} + {EI}_{parylene} \qquad ( 9 )$$

$$EI = {\sum }_{i = 1}^{N} {E}_{i} {t}_{i} [ {( b - {\sum }_{j = 1}^{i} {t}_{j} )}^{2} + ( b - {\sum }_{j = 1}^{i} {t}_{j} ) {t}_{i} + \frac{1}{3} {t}_{i}^{2} ] \qquad ( 10 )$$

$$where$$

$$b = \frac{{\sum }_{i = 1}^{N} {E}_{i} {t}_{i} ( {\sum }_{j = 1}^{i} {t}_{j} - \frac{1}{2} {t}_{i} )}{{\sum }_{i = 1}^{N} {E}_{i} {t}_{i}} \qquad ( 11 )$$

E₁ is the elastic modulus of the i^{th} layer.

The elastic energy of skin is represented as:

$${U}_{skin} = \frac{1}{{λ}_{rough}} {\int }_{0}^{{λ}_{rough}} \frac{{σ}_{z} {u}_{z}}{2} dx = \frac{π {{E}_{skin} ( {h}_{rough} - h )}^{2}}{16 {λ}_{rough}} \qquad ( 12 )$$

where the normal stress of the skin surface is:

$${σ}_{z} = \frac{π {E}_{skin} ( {h}_{rough} - h )}{2 {λ}_{rough}} cos \frac{2 π x}{{λ}_{rough}} \qquad ( 13 )$$

Adhesion energy is calculated as:

$${U}_{adhesion} = - γ {\int }_{0}^{{λ}_{rough}} \sqrt{1 + {( {w}^{′} )}^{2}} dx \approx - γ ( 1 + \frac{{π}^{2} {h}^{2}}{4 {λ}_{rough}^{2}} ) \qquad ( 14 )$$

U_{conformal}=0 when the conformal contact occurs. Substituting into energy equation and solving for γ produces:

$$γ = \frac{( \frac{{π}^{4} \cdot EI \cdot {h}^{2}}{{λ}_{rough}^{4}} + \frac{π \cdot {{E}_{skin} ( h - {h}_{rough} )}^{2}}{16 {λ}_{rough}} )}{\frac{{π}^{2} \cdot {h}^{2}}{4 {λ}_{rough}^{2}} + 1} \qquad ( 15 )$$

$$h = \frac{{E}_{skin} \cdot {h}_{rough}}{\frac{16 {π}^{3} \cdot EI}{{λ}_{rough}^{3}} + {E}_{skin}} \qquad ( 16 )$$

In the present disclosure, skin conditions and material properties are h_{rough}=25.7 μm, e=331.7 μm, Eₛₖᵢₙ=130 kPa, h₁=50 nm, A=6 mm², E₂=330×10⁹, and E₂=3.8×10⁹.

The inventors obtained the work of adhesion as a function of parylene thickness (t₂). The work of adhesion, as described by Eq. (15), is larger than the bending-induced strain energy per area of the electrode given by Eq. (1). Then, the bending-induced strain energy per area (represented by the solid line) and the work of adhesion (represented by the dotted line) are plotted in FIG. 1E. The inventors observed that the two values converge at a parylene thickness of about 300 nm when the electrode structure makes conformal contact, as shown in FIG. 1E. The inventors identified that below this thickness, conformal contact is theoretically achievable and for higher thickness mismatch of the topographies might happen. Therefore, in some embodiments, the inventors concluded that it was essential to maintain the parylene thickness thinner than 300 nm to ensure conformality to epidermal topography and reduce the delamination risks.

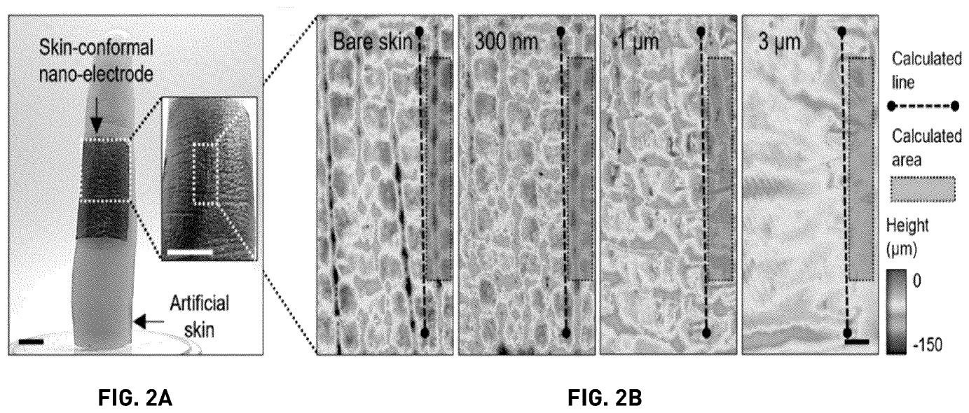

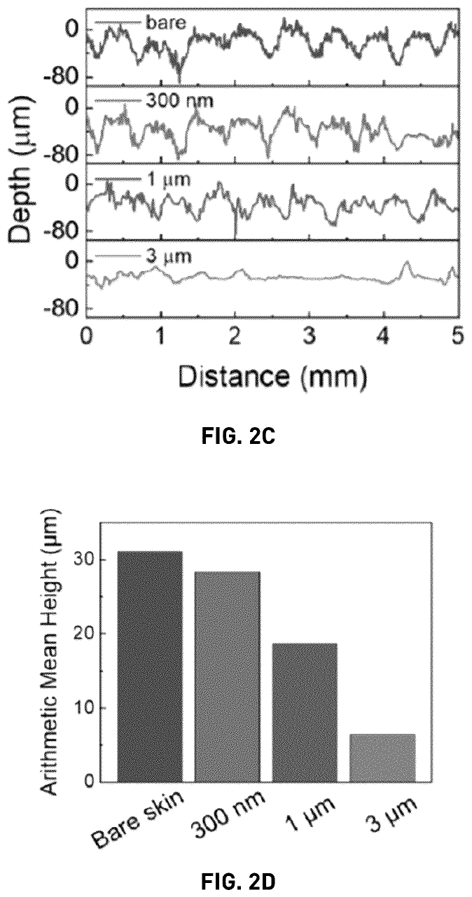

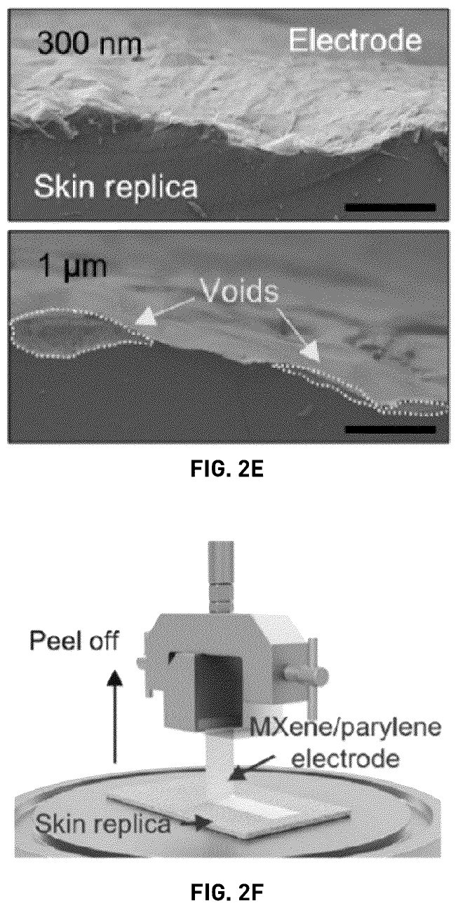

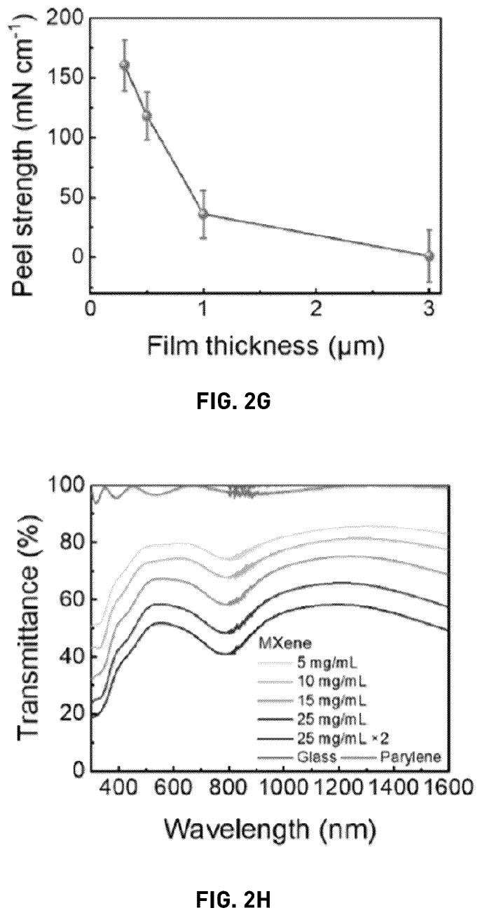

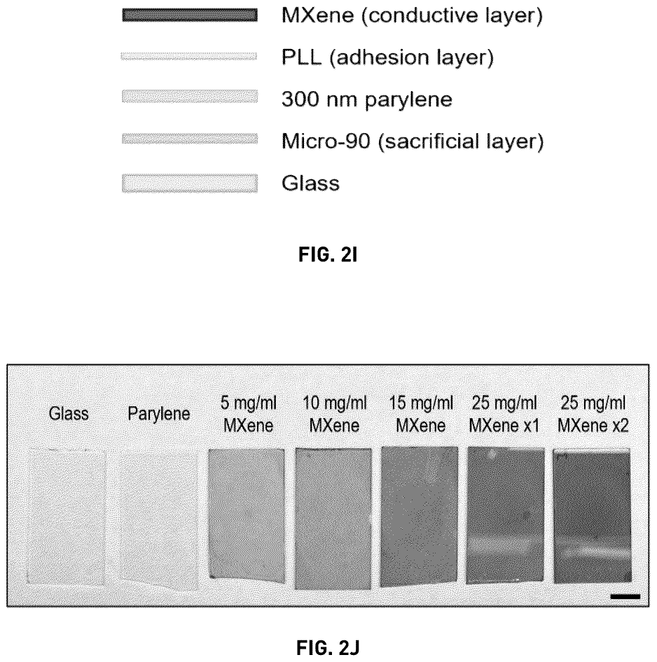

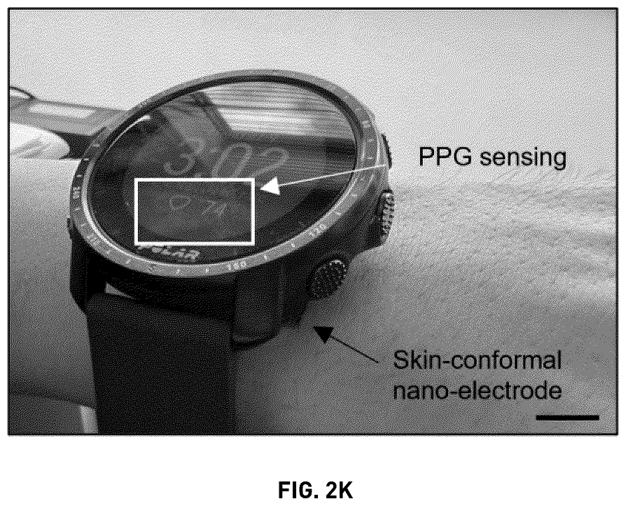

Structural Analysis of Conformal Coatings. The inventors investigated the conformability of the nano-electrodes on a skin replica and compared it with 1- and 3-μm-thick electrodes by using a profilometry height profile analysis with a three-dimensional (3D) laser microscopy (see FIG. 2A).

The profile image of the nano-electrode (right) exhibited highly conformal contact, closely following the bare skin (left) (see FIG. 2B). Moreover, along the dashed black line and gray area, inventors analyzed the height profile and 3D areal surface roughness analysis, respectively.

Indeed, the morphology of the skin replica covered with the nano-electrode is similar to that of the skin itself, while a notable decrease in conformal contact occurred in 1-, and 3-μm-thick electrodes leading to reduced adaptation to the skin textures in FIG. 2C. The inventors further analyzed the 3D areal surface roughness using an arithmetic mean height (Sa) parameter in FIG. 2D. The Sa values for bare skin and MXene/parylene electrodes with thicknesses of 300 nm, 1 μm, and 3 μm showed 31.1, 28.4, 18.7, and 6.5 μm, respectively. These analyses suggested that the nano-electrode exhibited superior conformability, even covering the deep valleys of the micro-rough fingerprint texture. Furthermore, the superior conformability of the 300 nm nano-electrodes on the artificial skin was confirmed with SEM (see FIG. 2E). This imaging confirms that the nano-electrode ensured highly conformal contact on curvilinear surface textures, whereas the 1-μm-thick and 3-μm-thick electrodes allow for microscopic air pockets (see FIG. 2E).

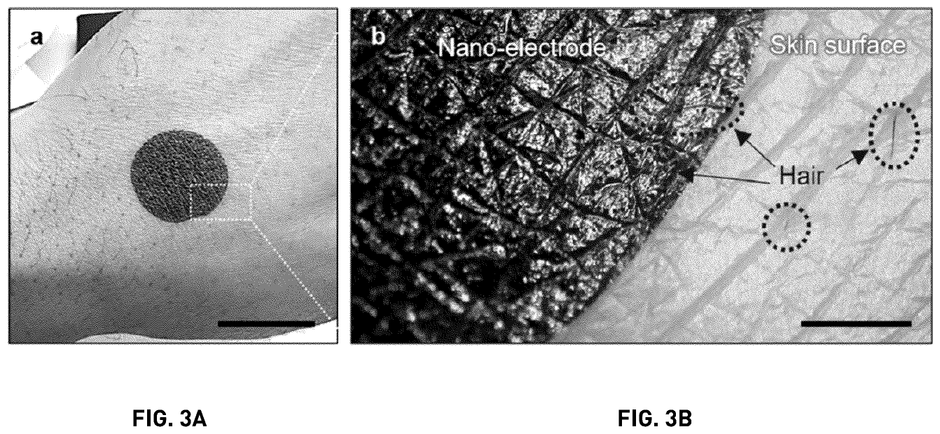

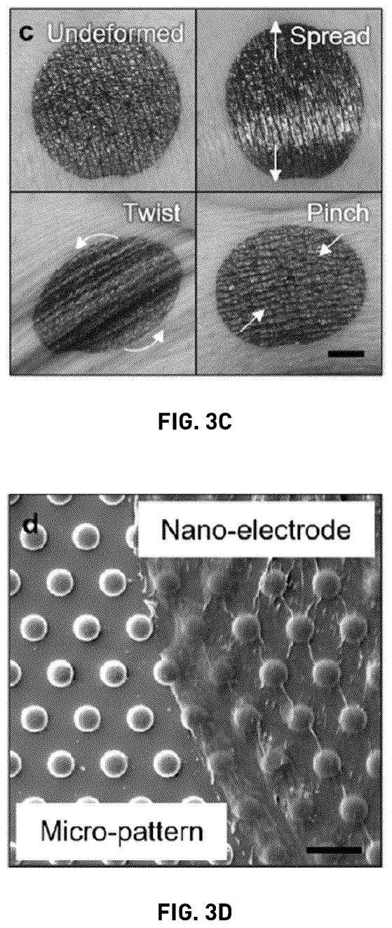

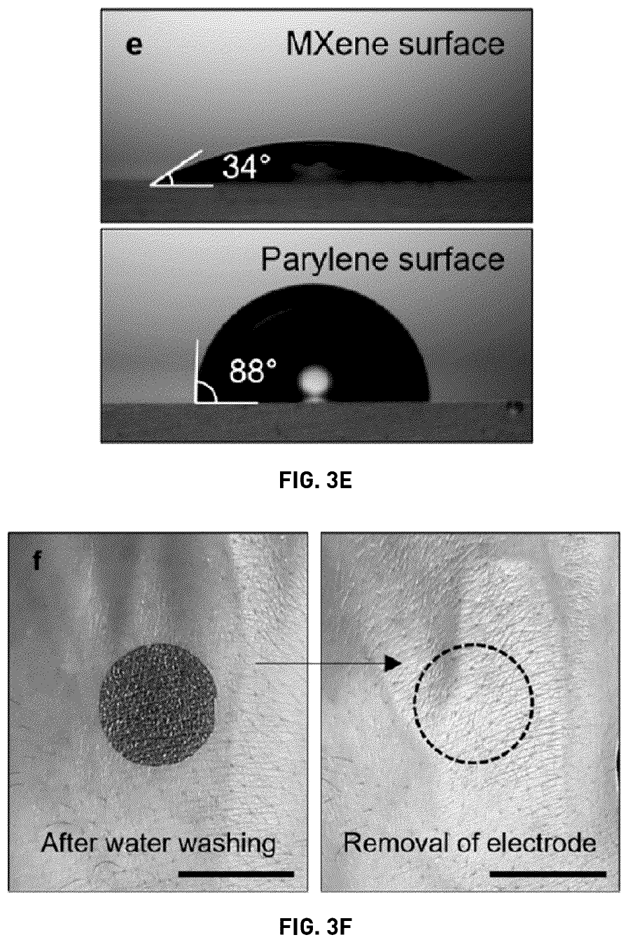

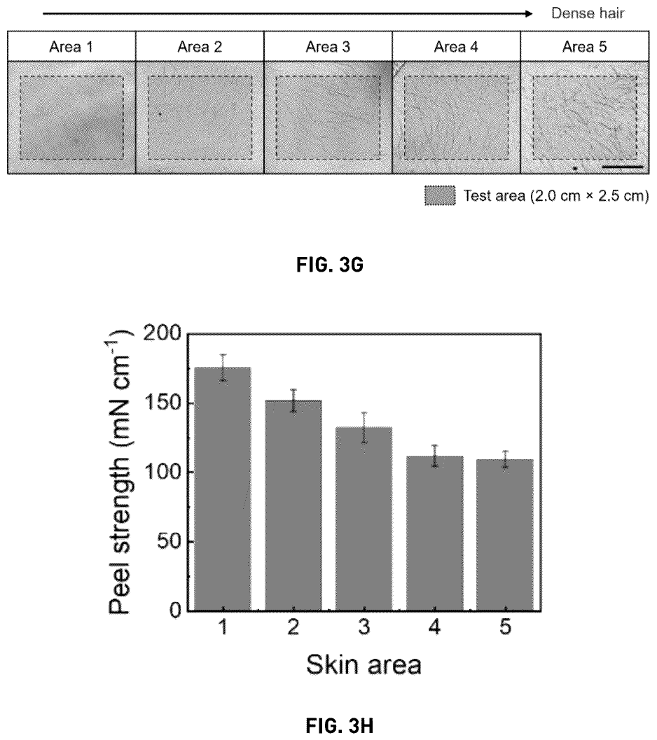

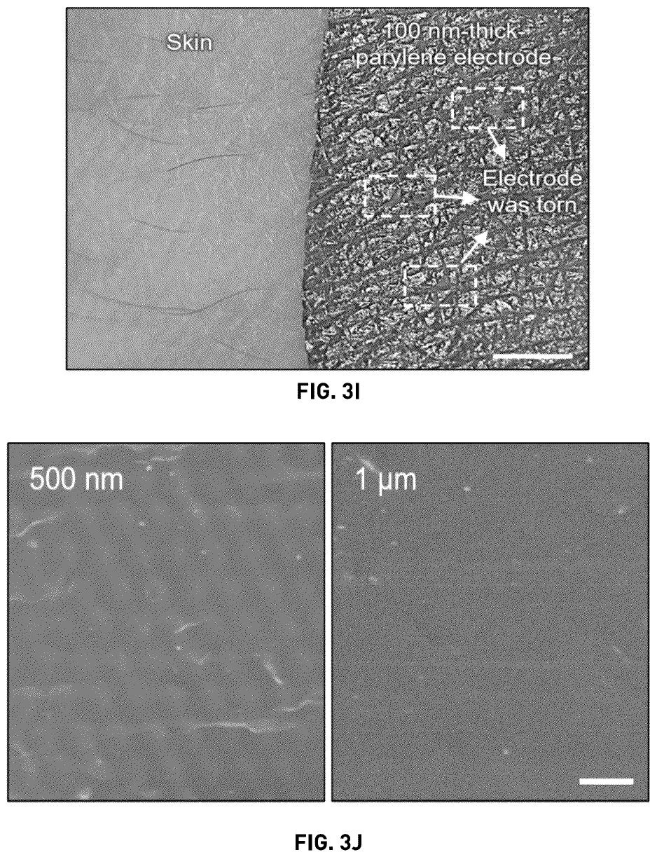

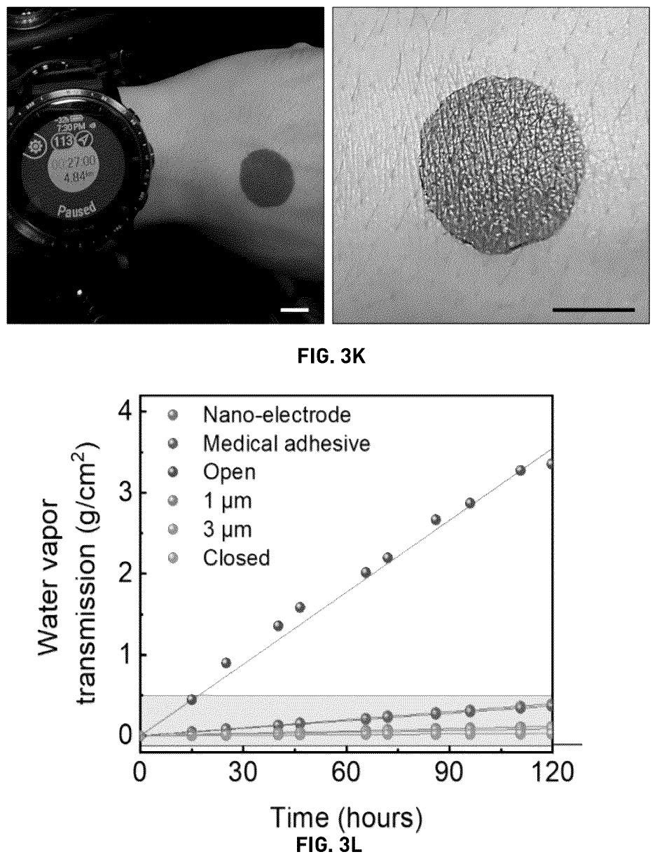

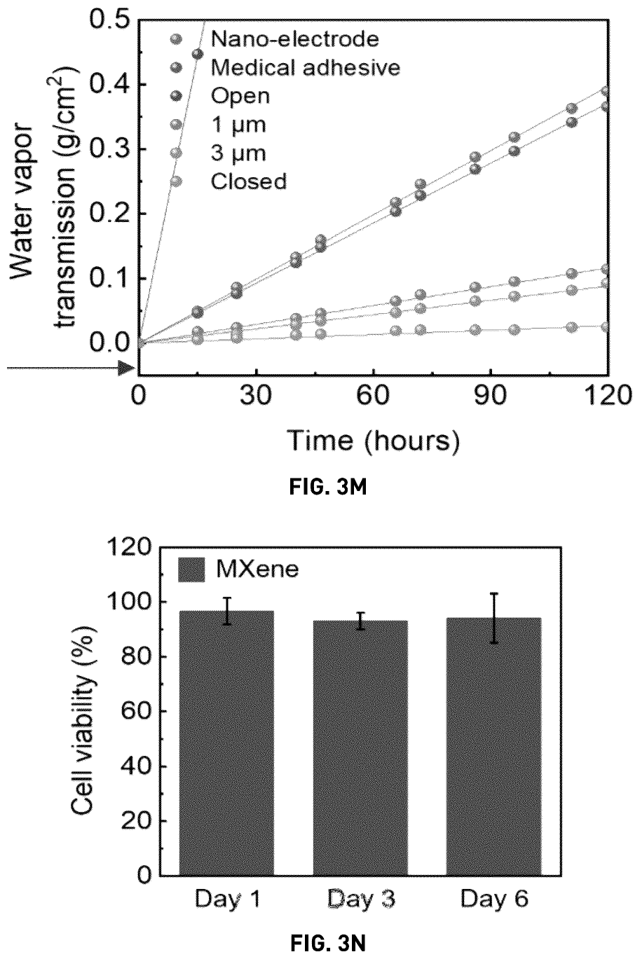

To further quantify the skin-electrode adhesion, the inventors conducted peel-off tests on four different electrodes (0.3-, 0.5-, 1-, and 3-μm-thick) on the commercial rubber artificial skin (see FIG. 2F). The inventors observed that the peel strength, calculated as force per unit width of the film, increased significantly, by two orders of magnitude with decreasing parylene thickness (see FIG. 2G). This dependence further confirms the role of the increasing contact area between the thinner electrodes and the skin. In FIG. 2H, the inventors observed a broad range of optical transmittance (T of 51.9-79.3% at 550 nm and 52.8-80.3% at 940 nm) depending on MXene concentrations (5-25 mg/mL)(see FIGS. 2I and 2J). These optical properties suggest potential for integration with multifunctional optical sensing applications such as photoplethysmography (PPG) or pulse oximetry. For example, as shown in FIG. 2K, a PPG sensor provided on a commercial heart rate monitoring watch was placed over the skin-conformal nano-electrodes, and the beats per minute (BPM) monitoring function using the PPG sensor on the commercial watch was not affected by the presence of the skin-conformal nano-electrodes between the watch and the skin. Nano-Electrodes as Skin-LocatedSensors. To confirm the adaptability and conformability of the skin-conformal nano-electrodes on the human skin, the inventors transferred them on the back of the hand in FIG. 3A. The enlarged image exhibited high conformality to the skin but also some adaptability on hairs(dashed blue line) (FIG. 3B). The inventors conducted adhesion tests on the skin with varying hair densities. The results showed that while adhesion modestly (around 30%) decreased as hair density increased, the nanoelectrode still maintained high adherence to the skin (see FIGS. 3G and 3H).

By comparison, a 100-nm-thick parylene electrode on the same skin surface can be easily torn especially on the highly strained valley of a fingerprint (see FIG. 3I). In contrast, skin-conformal nano-electrodes of 300 nm thick are extremely stable at common large skin deformations such as spreading, pinching, and twisting which facilitates stable signal collection and low-motion artifacts, as discussed below (FIG. 3C).

Independent testing of electrode assembly on dome-like patterned polydimethylsiloxane substrates imitating characteristic topographical skin dimensions showed that the nano-electrode completely covered all features (FIG. 3D). In contrast, thicker electrodes (0.5- and 1-μm-thick parylene) are suspended over surface features (see FIG. 3J), thus confirming that reaching the around 300-nm-parylene thickness predicted by theory is essential for full conformal coatings of microscopic topographical features. This conformal coating efficiently eliminates air pockets and increases the total contact area for electrode integration for enhanced sensing performance as described below. Moreover, the conductive MXene surface showed good wettability with a water contact angle of 34°, which should assist in electrode spreading and facilitate self-adhesion between electrodes and the skin surface (FIG. 3E). This low contact angle is similar to that of cleaned skin (typically 50° to 80°), which also exhibits better wettability, allowing water to spread more evenly. On the other hand, the outer parylene surface can repel the water droplet as indicated by a higher water contact angle of 88°, common for hydrophobic surfaces and oily skin (typically 900 to 100°) causing water to bead up rather than spread. This unique design of dual wettability allows for strong physical adherence of ultrathin nano-electrodes with dual hydrophobicity.

Using Equation (9) above, the bending stiffness of the nano-electrode is calculated to be 2.1×10⁻¹² N·m. The bending stiffness of skin (EIₛₖᵢₙ) can be calculated using the following equation:

$${EI}_{skin} = \frac{{E}_{skin} \cdot {t}_{skin}^{3}}{12 \cdot ( 1 - {v}^{2} )} \qquad ( 17 )$$

where EIₛₖᵢₙ=130 kPa, tₛₖᵢₙ=1.5 mm, and ν=0.48 is the Poisson's ratio of the skin. The resulting calculation is EIₛₖᵢₙ=3.8×10⁵ N·m, which is approximately 1.8×10⁷ times stiffer than the nano-electrode. As such, the bending stiffness (or flexural rigidity) of the nano-electrode (2.1×10⁻² N·m) is significantly lower than that of human skin (3.8×10⁻⁵ N·m) that facilitates conformal deformation of nanoelectrodes at stiffer substrates with larger radii of curvature during deformations.

The flexibility and low stiffness of the nano-electrodes allow for conformal contact to the skin's natural topography, particularly in dynamic environments, thus minimizing mechanical mismatch between the electrode and skin.

Such a combination of maximized contact area and matching wettability facilitates strong capillary facilitated physical adhesion of the nano-electrodes to the skin. Indeed, these electrodes sustain strong water flow and rubbing in water (FIG. 3F, left). Moreover, numerous skin deformations (>6,000 times) and sweating during approximately 5 km of running do not compromise electrode attachment (see FIG. 3K). However, the nano-electrodes can be easily removed by handwashing with soap, which delaminates the film by penetrating surfactants. Furthermore, physically-driven adhesion without the need for additional adhesive tapes prevents excessive skin irritation after electrode washing out (FIG. 3F).

Additionally, the inventors evaluated the breathability of the nano-electrode by measuring its water vapor transmission rate (WVTR) (see FIGS. 3L and 3M). The results show that the nanoelectrode exhibits a WVTR of 3.3 mg cm⁻² h⁻¹, which is comparable to common medical dressings (3.1 mg cm⁻² h⁻¹, Tegaderm, 3M Medical), enabling effective moisture exchange while maintaining strong skin adhesion. This level of breathability helps minimize sweat accumulation beneath the electrode during prolonged wear, thereby reducing and ensuring user comfort. To further evaluate the safety of prolonged skin contact, the inventors conducted cell viability tests, which revealed that after 1, 3, and 6 days of incubation, cell viability remained above 94%, confirming minimal cytotoxicity of the electrodes (see FIG. 3N).

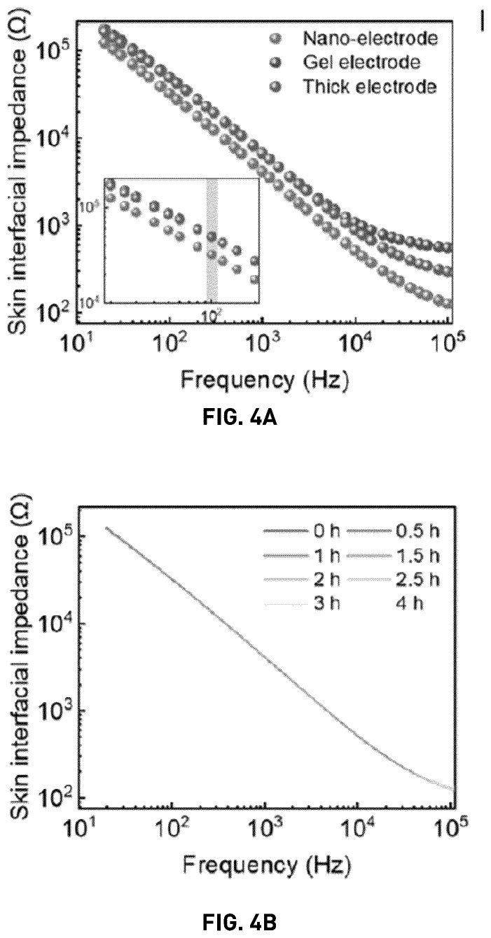

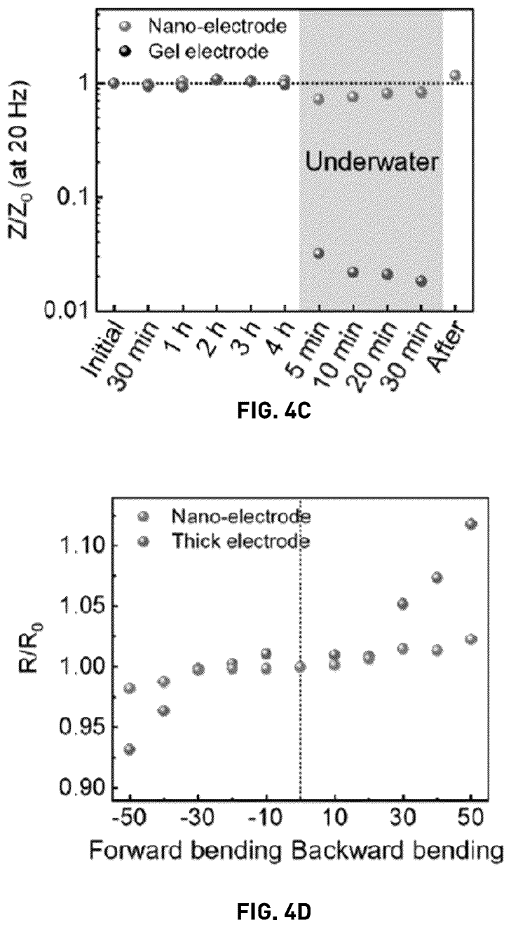

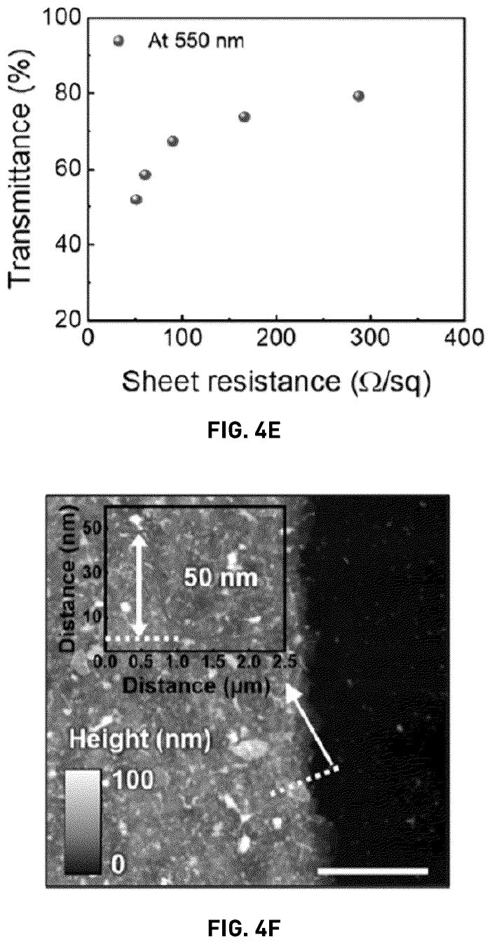

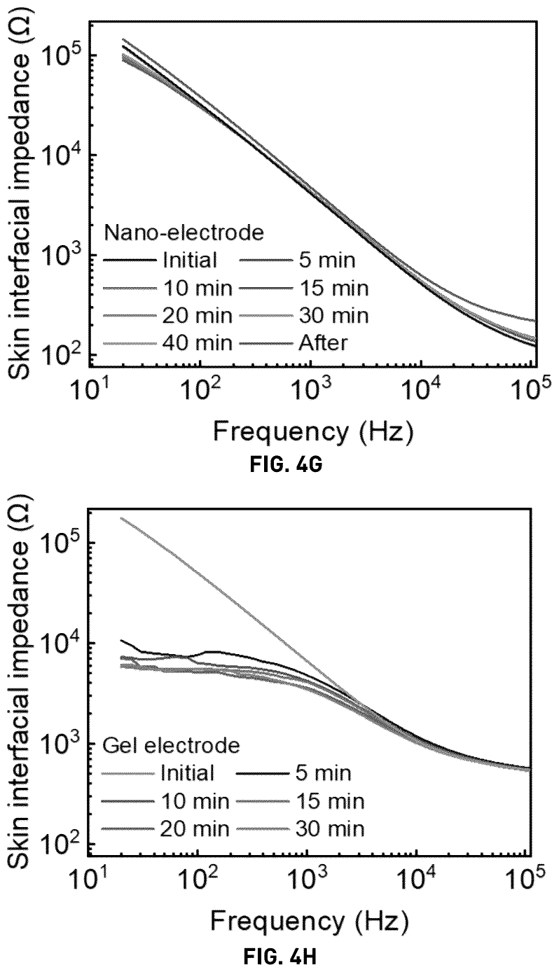

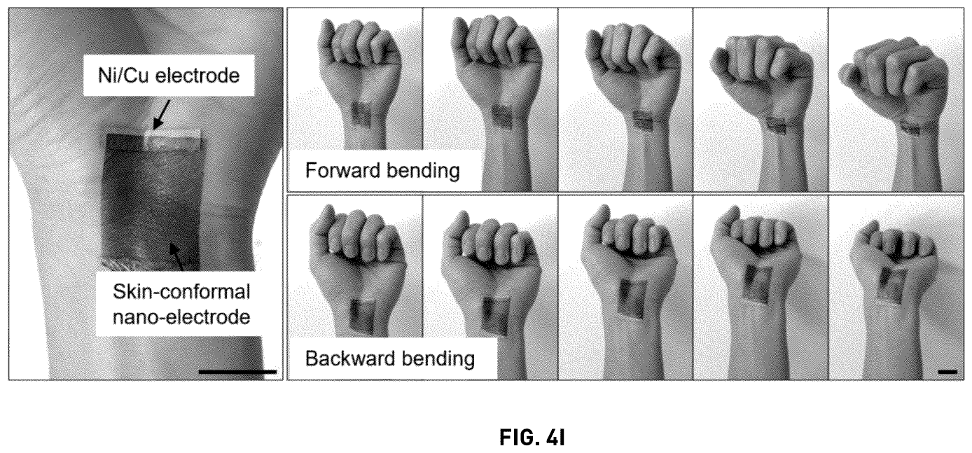

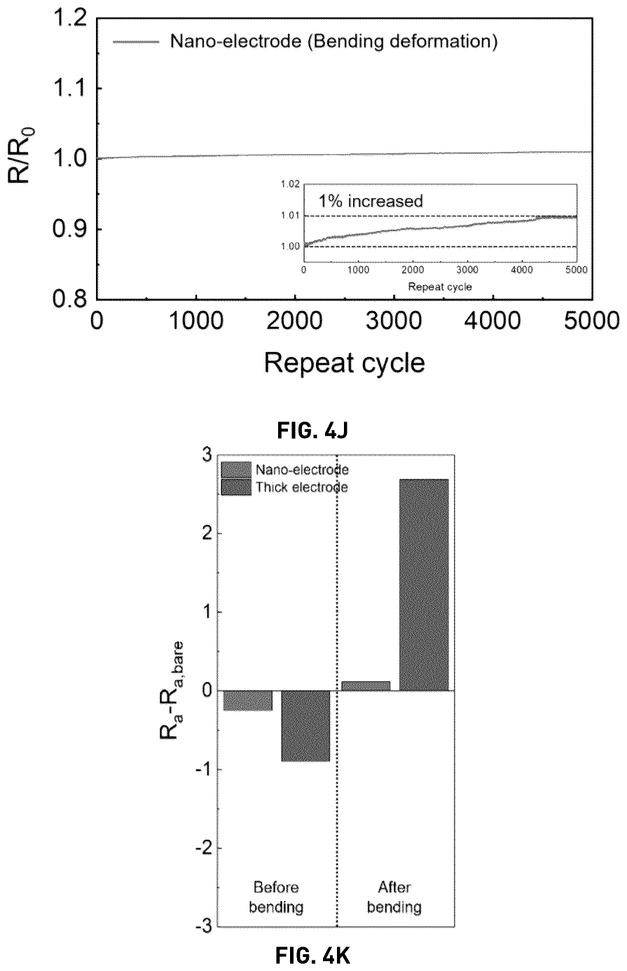

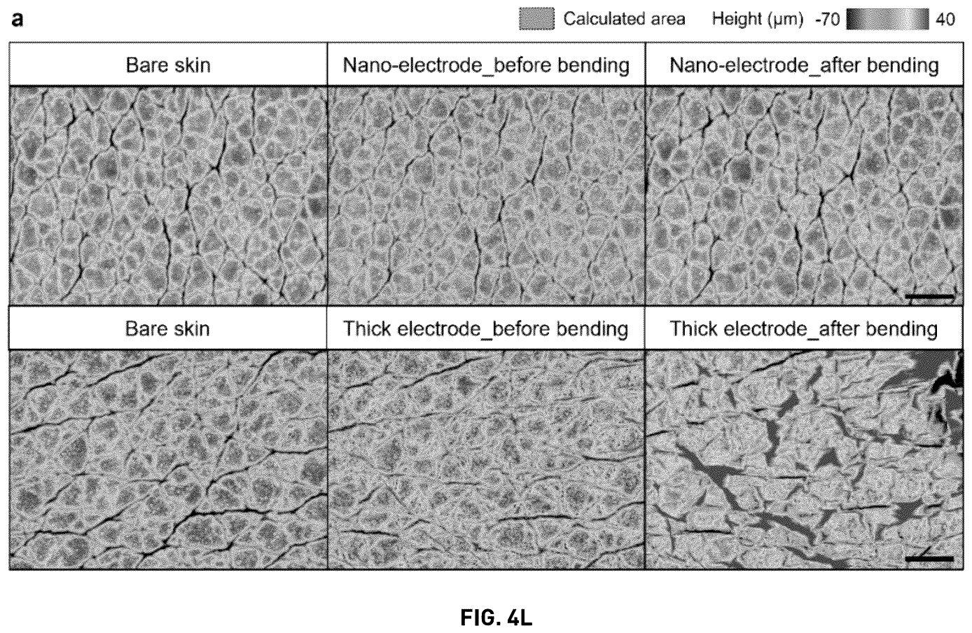

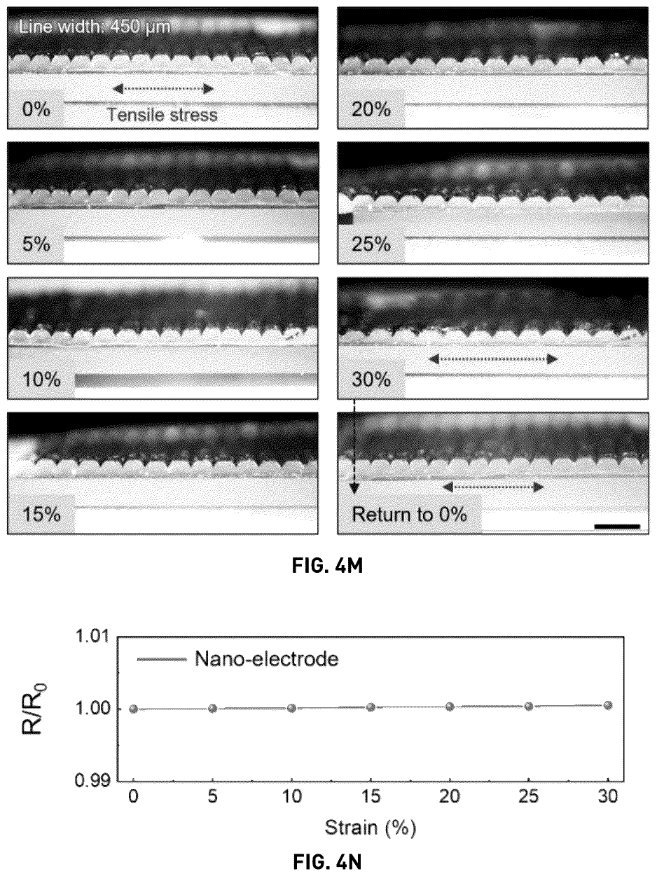

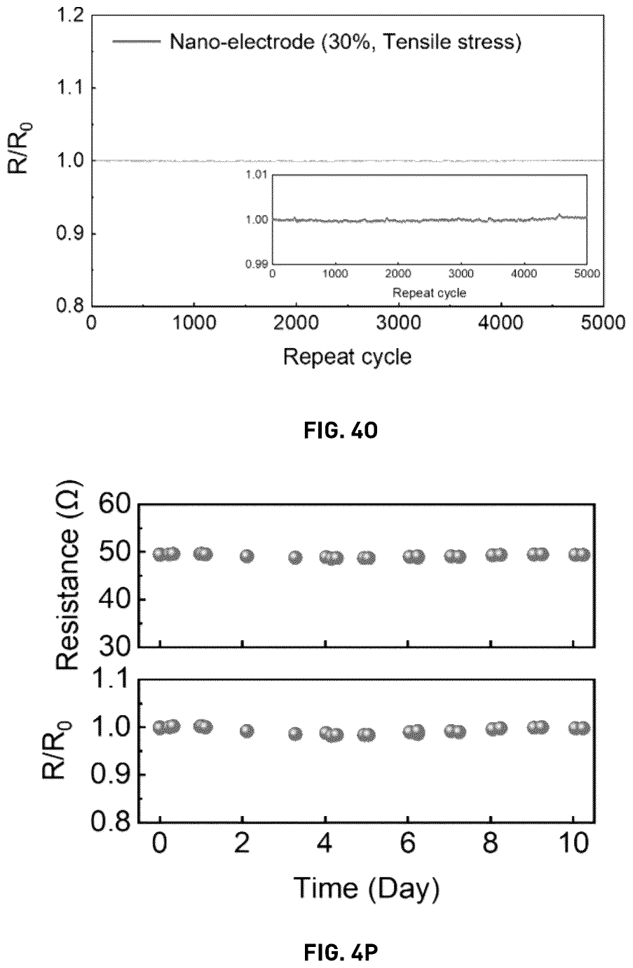

Electrical characteristics of the nano-electrodes. High conformability at the skin-electrode interface ensures the efficient transfer of electrophysiological signals to the electrode resulting in greatly increased signal amplitude, minimizing noise and signal attenuation, and reducing motion artifacts, all enhancements not achievable with traditional electrodes (see e.g., FIG. 4A). For electrical characterizations of skin-conformal nano-electrodes at the skin-electrode interface, the inventors placed three types of electrodes such as nano-, gel, and 1-μm-thick parylene (denoted as thick) electrodes on a volunteer's forearm.

The inventors observed that the nano-electrode showed a lower skin interfacial impedance (within 20 Hz to 100 kHz) than that of gel and thick electrodes (FIG. 4A). At 100 Hz, the impedance of the nano-electrode (32.3 kΩ) was much lower (about 35%) than that of the gel (50.2 kΩ) and thick (49.0 kΩ) electrodes (FIG. 4A, inset). It is noteworthy that the low impedance of the nano-electrodes (compared to that of thick electrodes) is due to the skin-electrode contact instead of the conductivity of the electrodes (the sheet resistance of nano- and thick electrodes was 50 Ωsq⁻¹). In addition, due to the high conformability, skin-conformal nano-electrodes exhibited long-term impedance stability over a period of 4 hours in FIG. 4B.

The inventors further evaluated the underwater electrical properties of the skin-conformal nano-electrodes (FIG. 4C). The nano- and gel electrodes were immersed in the water following long-term measurements and the skin interfacial impedance was measured underwater for 30 minutes (see FIGS. 4G and 4H). The impedance at 20 Hz of gel electrodes significantly decreased with increased immersion time, with the relative impedance dropping to 0.018 after 30 minutes of immersion. The reduction in impedance was attributed to water absorption and gel swelling.

After underwater analysis, the gel electrodes lost adhesion to the skin, preventing any further impedance measurements, and highlighting a significant limitation of gel electrodes in water-exposed environments. In contrast, the skin-conformal nano-electrodes maintained stable impedance both in air and underwater, particularly following underwater measurements, indicating that electrophysiological signal acquisition is less affected by external environmental conditions. This is attributed to high conformability at the skin-electrode interface and the control of the dual hydrophobicity with low wettability of the parylene side and the high wettability of the MXene side.

To evaluate the electrical properties of nano- and thick electrodes in response to forward and backward bending, the inventors attached them to the inner wrist with Ni/Cu tape connected to the sides of the electrode (see FIG. 4I). As shown in FIG. 4D, the relative resistance changes of the nano-electrode were only ±2% for forward bending and backward bending. In contrast, the thick electrode exhibited relative resistance changes of −7% for forward bending and 12% for backward bending. The minimal relative resistance changes observed in the nano-electrode, as well as high skin-electrode adhesion, affect low-motion artifact and high-fidelity electrophysiological signal acquisition.

The inventors further assessed the nano-electrode's electrical durability under repeated 5,000 bending cycles on artificial skin, observing high stability and only about 1% relative resistance alternation over long cycling (FIG. 4J). Moreover, the height profile analysis also confirmed that the nano-electrode maintained firm conformal contact even after 1,000 bending cycles, with no significant changes in surface topography after repeated dynamic stresses (FIGS. 4K, 4L).

To evaluate electrical stability under tensile stress, the nano-electrode was transferred onto a line patterned silicone elastomer substrate as a deformable model substrate (Ecoflex 00-30, line width: ˜450 μm) while pre-stretched by 30% in order to evaluate conformal adhesion and structural integrity over the patterned surface (see FIGS. 4M, 4N, 4O). After nano-electrode deposition, the inventors measured relative resistance changes under tensile strain and observed exceptional stability, with negligible variation even at high 30% strain and after 5,000 stretching cycles.

In addition, the low sheet resistance of the conductive MXene layer (50.9-287.3 Ωsq¹) allows for reliable electrophysiological signals and reduced noise interference during both static and dynamic conditions and maintains a certain level of optical transmittance (with a transmittance of 51.9-79.3% at 550 nm) as shown in FIG. 4E. Additionally, the nano-electrodes exhibited long-term stability with a negligible resistance change over 10 days (see FIG. 4P).

As shown in FIG. 4F, the high electrical stability of the nano-electrode, combined with its strong skin-electrode adhesion, enables low-motion artifact and long-term, high-fidelity electrophysiological signal acquisition due to the ultrathin (50 nm) MXene layer. Overall, skin-conformal nano-electrodes disclosed herein here can be configured into the curvilinear surfaces of the skin while maintaining high capacitance and low impedance, allowing for reliable detections such as ECG and EMG signals.

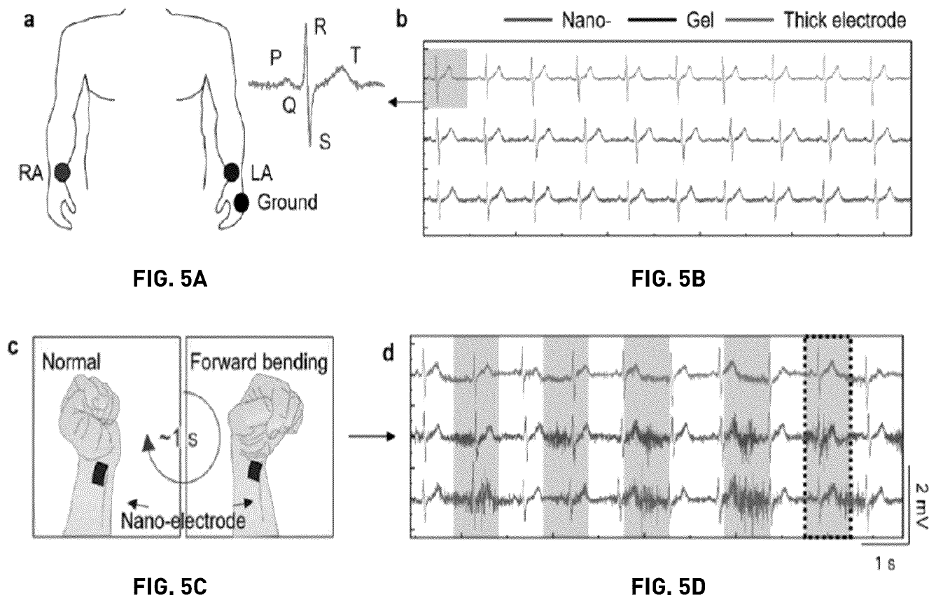

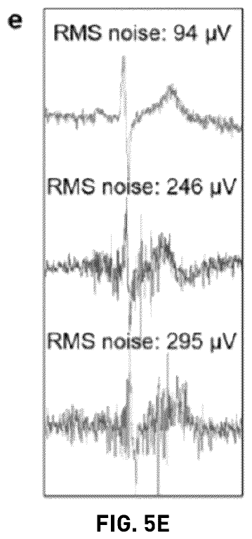

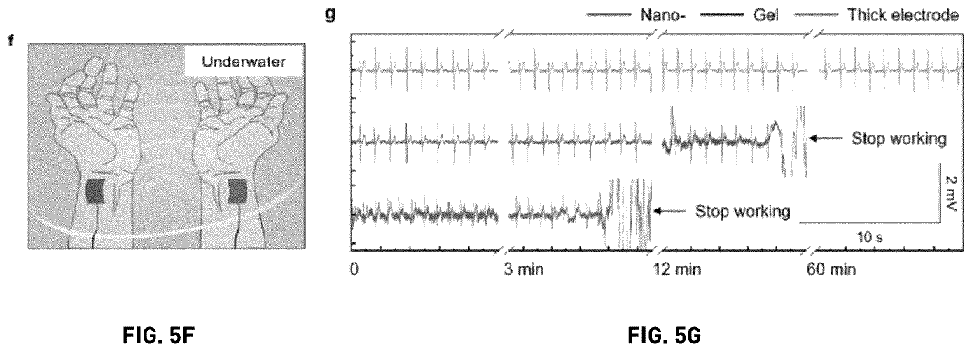

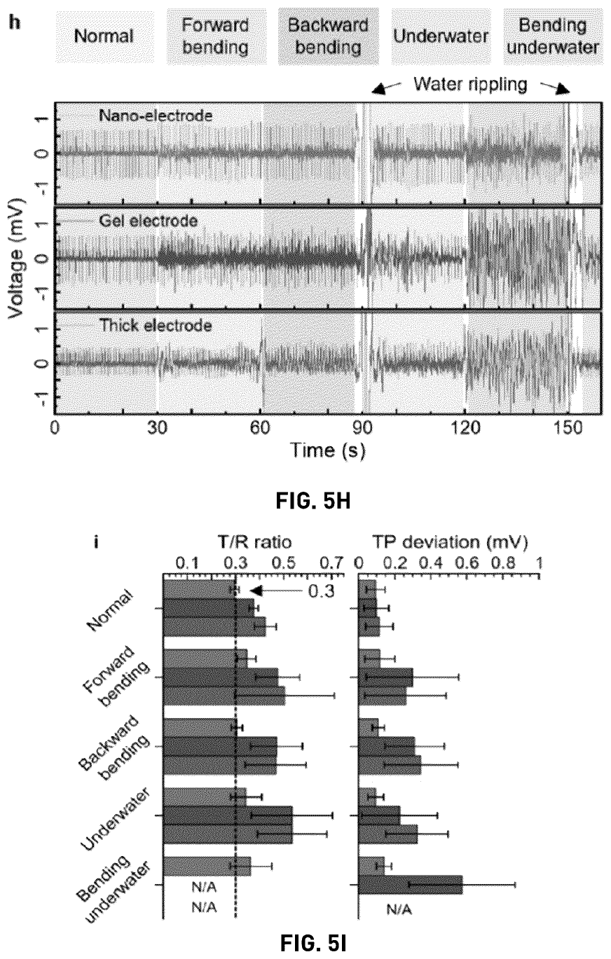

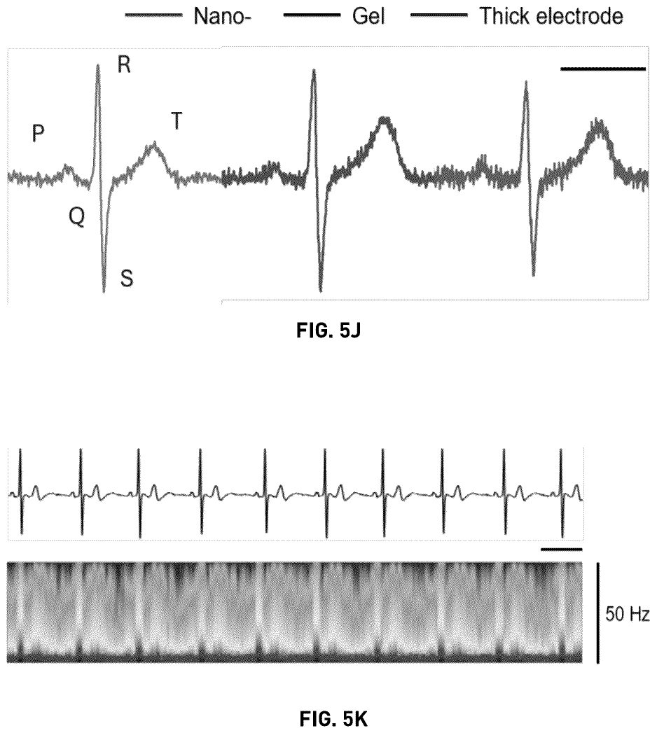

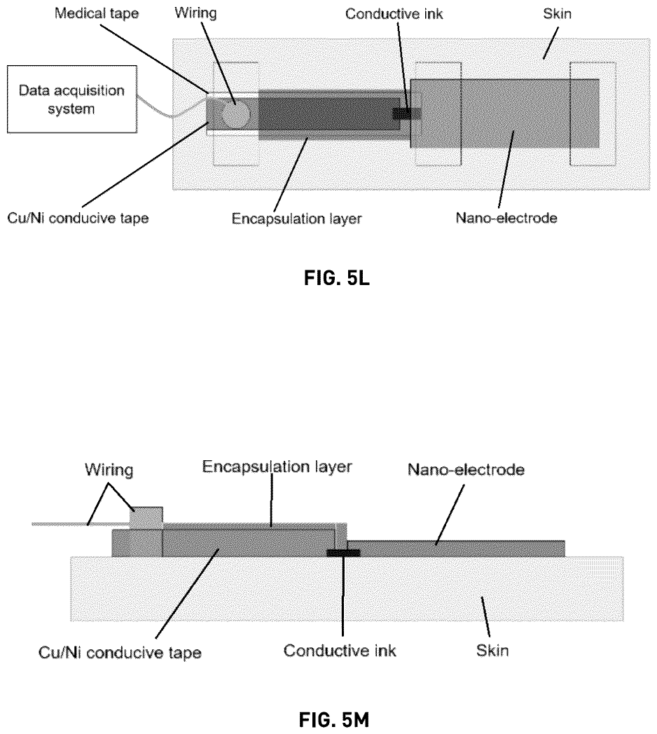

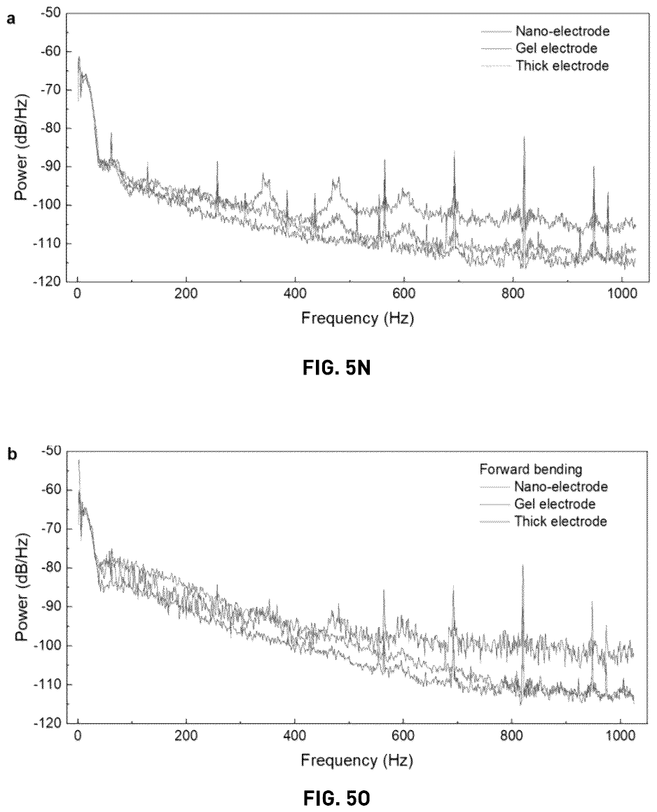

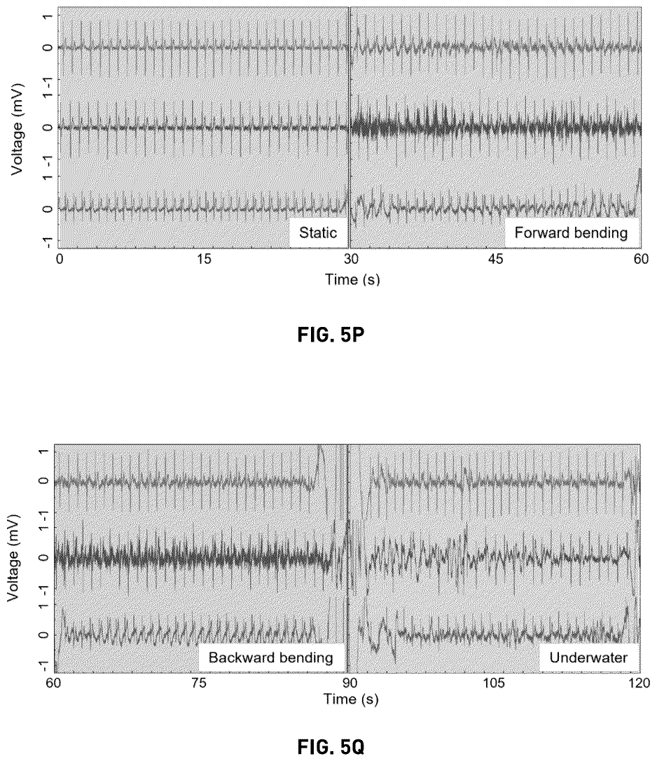

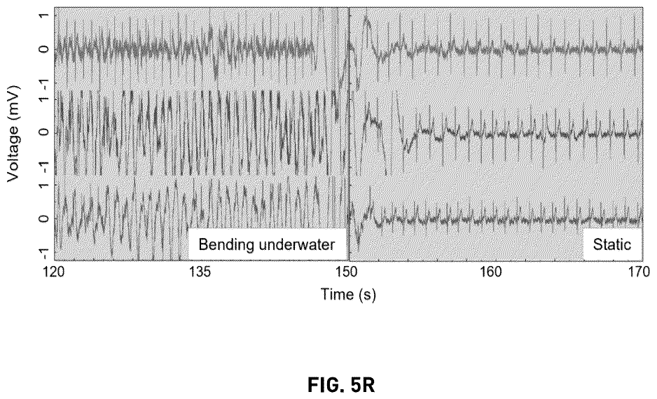

ECG analysis for low-motion artifacts under variable conditions. In the three-electrodes system employed in this study, electrodes were placed on the wrists of the right (RA) and left arms (LA), and on the back of the right hand as the ground electrode (FIG. 5A). ECG signals are composed of several peaks, collectively forming the PQRST waveform, which represents one complete heartbeat cycle, including the P wave, the QRS complex, and the T wave. Each component corresponds to different phases of heart activity, such as atrial and ventricular depolarization and repolarization, for identifying cardiovascular conditions.

As shown in FIGS. 5B, 5J and 5K, the ECG signals of the nano-electrodes demonstrated higher performance compared to gel-electrodes and thick electrodes. The nano-electrodes exhibited a superior SNR (35.1 dB), peak-to-peak voltage (1.40 mV), and RMS noise (32 μV), characterized by up to 30% increase in SNR, up to 25% increase in peak-to-peak voltage, and reduced noise by 30-60%, compared to the gel and thick electrodes. In addition, the time-dependent voltage signals, and corresponding short-time Fourier transform spectrogram up to 50 Hz of the nano-electrodes allowed for better localization of transient events and the detection of subtle changes in frequency components over short-time intervals identifying and characterizing abnormalities or irregularities in arrhythmias or ischemic events.

ECG signals were detected during wrist movements, as shown in FIG. 5C. The inventors repeatedly performed a normal state and forward bending movement at 1-second intervals. To minimize motion artifacts at the interface between the nano-electrode and the interconnected electrical lead, the inventors developed a device incorporating a mechanical gradient design (see FIGS. 5L and 5M). In FIG. 5D, the ECG signals of the nano-electrode were recorded with consistent and clear PQRST peaks under a normal state and forward bending (indicated by the gray blocks), indicating high-fidelity ECG signals with low-motion artifacts.

However, the ECG signals of gel and thick electrodes made it hard to distinguish PQRST peaks. The RMS noise of the nano-electrode during motions showed a relatively low change (94 μV), whereas the gel and thick electrodes exhibited 2.6- and 3.1-times higher RMS noise respectively, which coincides with the noticeable noise increase in FIG. 5E. The increased noise level of the gel and thick electrodes is attributed to shearing at the gel-skin interface (see FIGS. 5N and 5O). To test the underwater stability further, the inventors attached electrodes to the same location and then completely immersed them in water for 60 minutes while exercising different activities (FIG. 5F).

As observed, the common gel and thick metal and polymer electrodes were detached from the skin almost immediately, within a few minutes, completely destroying the measuring routine. In contrast, the ECG signals of the nano-electrode were consistent and clear PQRST peaks were observed at the longest testing times within an hour (see FIG. 5G). The conformal hydrophobic parylene layer prevents water penetration at the skin-electrode interface, enabling sustainable ECG signal monitoring with low-motion artifacts.

ECG analysis during intense physical activities. To elucidate the quality of continuous monitoring of ECG signals during wrist movement both in air and underwater (in the order of normal, forward and backward bending, underwater and forward bending underwater at 30-second intervals), the inventors measured the ECG signals of nano-electrodes, gel-electrodes, and thick electrodes for comparison (see FIG. 5H, with enlarged views of the same data shown in FIGS. 5P, 5Q, 5R). The applied repeated physical activities (repeatedly performed a normal state and forward/backward bending movement at 0.5-second intervals) generally introduced motion artifact ECG signals, particularly in gel and thick electrodes. Nonetheless, the recorded ECG signals using the nano-electrode demonstrated superior performance, exhibiting reduced noise and consistent signal quality under all tested conditions.

In FIG. 5I, the inventors introduce two parameters, such as T/R ratio and TP deviation to analyze ECG signals for assessing the heart's electrical activity and providing insights into various cardiac conditions. First, the T/R ratio is the ratio of the amplitude of the T wave to the amplitude of the R wave. The T wave represents ventricular repolarization, while the R wave represents ventricular depolarization. It is typically considered to be around 0.30±0.12 in good-quality ECG signals. Accurate monitoring of the T/R ratio is crucial, as an abnormal value (0.18±0.16) may indicate certain cardiac conditions, including ventricular fibrillation. The nano-electrode maintained a T/R ratio close to 0.3 across all physical activities. However, the T/R ratios for the gel and thick electrodes were higher than this ideal range. Notably, the recorded ECG signals using these electrodes during bending underwater were difficult to interpret as original ECG signals.

Furthermore, the inventors analyzed a TP deviation, which is the deviation of the baseline between T and P waves in the ECG signal. More consistent TP deviations indicate more reliable ECG signal acquisition. In a normal state, all electrodes exhibited similar TP deviations. While the nano-electrodes maintained stable TP deviation values (0.95-1.41 mV) even under forward/backward bending and underwater conditions, the gel and thick electrodes exhibited significant increases in TP deviation, leading to unreliable ECG signals. This is due to the motion artifacts, poor skin-electrode contact, and water absorption/penetration. It is noteworthy that the superior stability and reliability of nano-electrodes in diverse conditions, ensuring reduced motion artifacts and effective water-resistant signal monitoring.

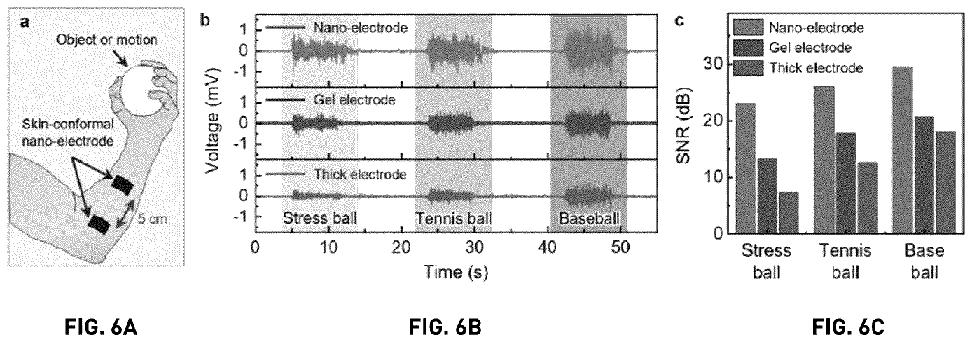

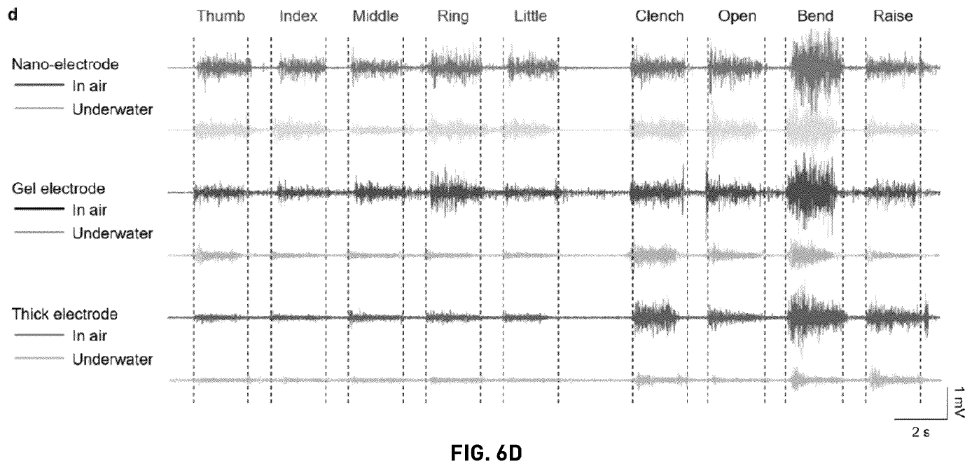

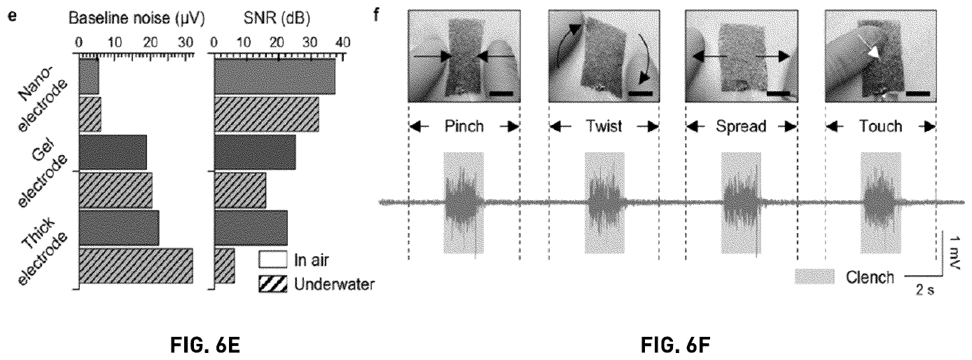

EMG monitoring under different conditions. For the acquisition of EMG signals from the brachioradialis muscle, the inventors mounted the electrophysiological electrodes on the forearm, and the back of the hand as the ground electrode (FIG. 6A). The inventors conducted an object-grasping experiment using balls with different resistances (in increasing order: stress ball, tennis ball, and baseball). To quantify and analyze the quality of ECG signals from various electrodes, the inventors first obtained the power spectral density estimate using Welch's method in MATLAB (‘pwelch’ command). The parameters for the pwelch command were set to a 2000-point Hanning window with 50% overlap. The frequency range of interest for ECG signals is typically below 100-120 Hz. To remove baseline wandering, the inventors filtered out frequencies below 0.5 Hz. As shown in Figure S11, the region between 0.5-100 Hz (highlighted in blue) was considered to represent the collected signal in our quality analysis. The inventors extracted noise from the region shown in red (100-1000 Hz), chosen because it lies explicitly in the noise floor. The noise level was then quantified using the root-mean-square value.

For EMG signals, the inventors used the same ‘pwelch’ command to analyze the quality of the EMG signal from the gripping test as the ECG signal above. To remove baseline wandering, the inventors filtered out frequencies below 10 Hz for the EMG signal. Unlike the ECG signal, a subject can control the EMG signal. The signals from the idle state (when the subject does not give any activity on muscle) were considered as noise and root-mean-squared for further analysis. Then, the following formula was used to convert the ratio of the signal and noise to power in dB:

$$SNR = 20 log ( \frac{{V}_{rms , signal}}{{V}_{rms , noise}} ) \qquad ( 19 )$$

where V_{rms,signal} is the RMS value of the signal, and V_{rms,noise} is the RMS value of the noise.

FIG. 6B shows that a higher modulus ball required greater force to grasp. The SNR of nano-electrode showed high signal intensity for different stress motions characterized by a significant (up to 3 times) increase as compared to conventional gel-electrodes and thick electrodes.

Next, the inventors measured EMG signals of the finger (thumb, index, middle, ring, little) and hand movements (clench, open, bend, and raise) both in air and underwater conditions at 2-second intervals (FIG. 6D). To verify the water absorption/penetration to electrodes underwater, the EMG measurement was conducted after immersing the electrodes underwater for 5 minutes. The results confirmed the water-resistant EMG signals of the nano-electrodes, as they maintained stable and reliable EMG signal acquisition even after immersion. This demonstrated the enhanced durability and functionality of the electrodes in both air and underwater conditions. While the current single channel configuration limits the discriminability of fine finger movements due to overlapping muscle activations, this limitation is not intrinsic. Rather, it highlights the potential for enhanced gesture recognition when integrated into multielectrode systems, providing more sophisticated and robust EMG-based human-computer interfaces.

The inventors further analyzed the baseline RMS value and SNR of bending motion (FIG. 6E). The EMG signals of the nano-electrodes demonstrated high consistency both in air and underwater. Specifically, the baseline RMS value for the nano-electrodes (5.5 and 6.1 μV) was lower by 22-29% in air and 19-30% in underwater conditions. Furthermore, the SNR for the nano-electrodes (37.7 and 32.5 dB) was 1.5-3.1 times higher in air and 1.5-5.2 times higher in underwater conditions compared to gel and thick electrodes. The greater performance differences, beyond the 35% lower skin interfacial impedance, stem from the nanoelectrode's skin conformability and adhesion in dynamic conditions, ensuring stable impedance, reduced motion artifacts, and enhanced signal quality.

The skin-conformal nano-electrodes demonstrated consistent EMG signals during clench motions under various deformations including pinch, twist, spread, and touch, ensuring accurate and reliable EMG signal acquisition and practical applications in dynamic environments (FIG. 6F). This consistency is attributed to baseline stability under the deformations. Therefore, the skin-conformal nano-electrodes provide reliable monitoring of muscle activity in diverse environments critical for versatile wearable technology.

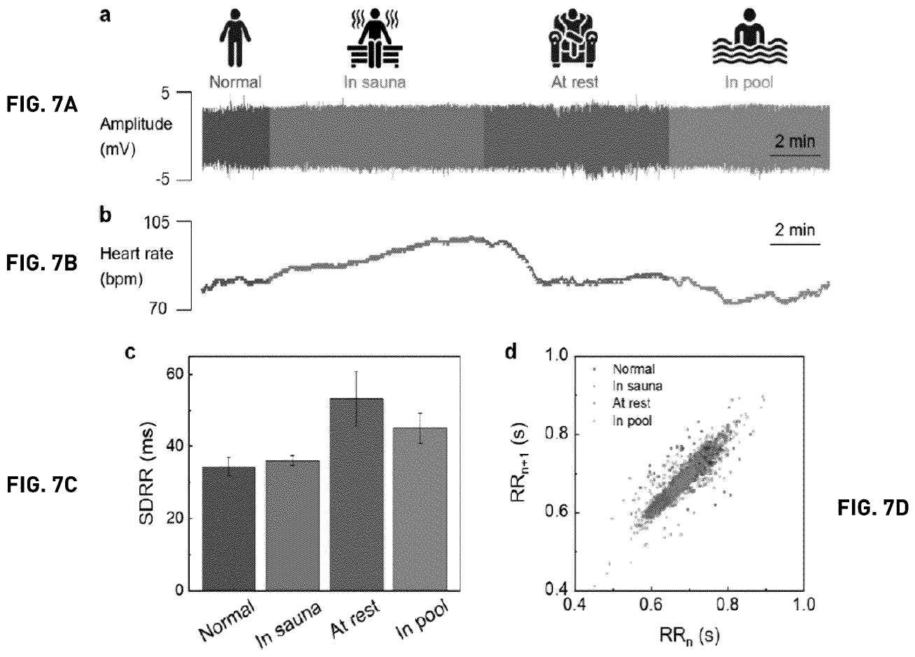

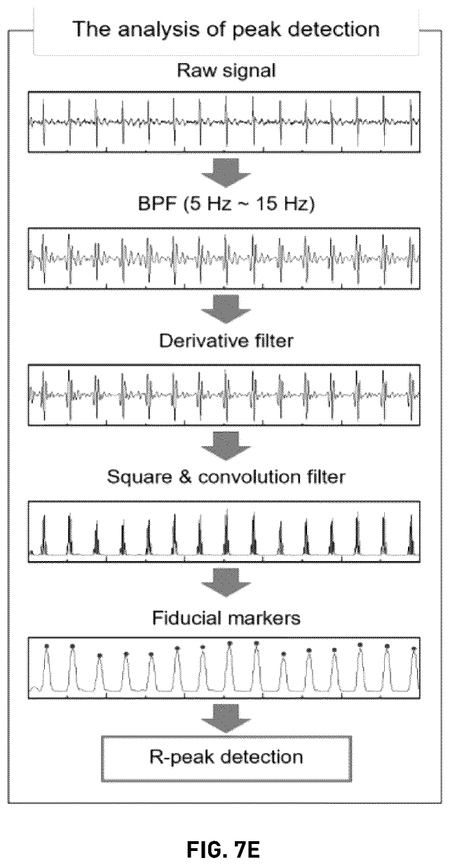

Heart rate monitoring in different testing environments. The skin-conformal nano-electrodes were used to monitor continuous ECG signals at extreme conditions such as a sauna and a pool (FIG. 7A). Based on the previous results in diverse environments, the other electrodes were unable to withstand the following conditions. ECG data was collected over 25 minutes, encompassing normal conditions, in a sauna, at rest, and in a pool. This comprehensive testing sequence was designed to assess the skin's ability to reliably capture ECG signals.

For the heart rate monitoring experiment, real-time ECG data was collected over a 25-minute period to observe heart rate changes under various conditions. The experiment consisted of four phases—Normal (2 minutes), In sauna (10 minutes), At rest (7 minutes), and In pool (6 minutes). In the first phase, after the mounted ultrathin electrodes completely dried, the participant sat on a chair at room temperature to establish a baseline heart rate in idle conditions. Heart rate in this state is referred to as ‘Normal’. Next, the volunteer entered an 80° C. dry sauna room within 10 minutes. This phase aims to observe the increase in heart rate due to heat stress. Following the sauna, in ‘At rest’ condition, the volunteer returned to the same conditions as in the ‘Normal’ phase for 7 minutes. This allowed the heart rate to decrease and return to a normal state. Finally, the volunteer entered a pool with a water temperature of 30° C. for 6 minutes to decrease heart rate due to the calming effect of water immersion. The experiment was designed to study how the volunteer's heart rate responded to different environmental conditions, including high-temperature heat stress and a comfortable swimming environment.

The recorded ECG data was subsequently processed to determine heart rate (HR) and heart rate variability (HRV), using the Pan-Tompkins algorithm, a widely recognized method for detecting QRS complexes in ECG signals (see FIG. 7E). This algorithm effectively highlights the R-peaks, the most prominent features of the ECG waveform, while reducing interference from other signal components. Initially, a digital band-pass filter (5 to 15 Hz) is applied to the raw signals to eliminate not only the baseline wandering, but also the high-frequency noise. The derivative filter emphasizes the rapid voltage changes of the QRS complex in ECG signals. It works as a high-pass filter, amplifying the steep slopes of the QRS waves while attenuating the lower-frequency P and T waves.

Furthermore, the filter enhances the distinction between the QRS complex and other undesirable signals. The following square and convolution steps integrate the output from the derivative filter. This step smooths out false peaks that may occur within a single QRS complex and creates a waveform that more accurately represents the QRS complex's shape and duration. With fiducial markers in place—ensuring precise timing, minimizing detection errors, and providing consistent reference points—the inventors can reliably detect continuous R-peaks.

Based on the R-peak detection using the Pan-Tompkins algorithm, the inventors calculated the dynamic response of HR encompassing normal conditions, in a sauna, at rest, and in a pool (see FIG. 7B). In a normal state, the HR remained steady at around 80 bpm. Upon entering the sauna, the HR gradually increases, reaching approximately 100 bpm. After leaving the sauna and entering a resting state, the HR rapidly decreases at first, then stabilizes at around 80 bpm. Subsequently, in the pool, the HR further decreases to about 73 bpm.

To elucidate the HRV analysis, which is a measure of the irregularity or variation between consecutive heartbeats, FIG. 7C exhibits the calculated standard deviation of RR intervals (SDRR) values for various conditions. RR interval is the time duration between two consecutive R waves in ECG signals and the SDRR is a measure of those variations in time RR intervals. Specifically, the SDRR values of normal, in sauna, at rest, and in pool exhibited 34.4, 36.1, 53.2, and 45.1 ms, respectively. These SDRR values across all circumstances fall within the normal range, which is typically 41.34±14.53 ms.

FIG. 7D presents a Poincare plot, a graphical tool commonly used in the analysis of HRV. The Poincare plot is a scatter plot of RR intervals, where each interval (RRₙ, x-axis) is plotted against the following interval (RRₙ₊₁, y-axis). The plot's shape and dispersion can offer both a visual and quantitative assessment of HRV by plotting consecutive RR intervals in four different conditions (normal, in sauna, at rest, in pool). The overall shape of the Poincare plot was elliptical, which is a general characteristic of a healthy heart. This elongated elliptical shape indicates regular variability in RR intervals, reflecting normal heart rate dynamics. Therefore, the analysis of HR and HRV in different environments demonstrates the robustness of the skin-conformal nano-electrodes in practical, real-world conditions.

Moreover, the accurate and stable acquisition of R-peaks across diverse environmental conditions not only supports basic HR tracking but also enables a wide range of potentially clinically meaningful analyses. In particular, HRV derived from RR intervals has been widely adopted to assess autonomic nervous system function, mental stress levels, cardiovascular risk, and even sleep quality. These results highlight the potential of ultrathin skin-conformal nano-electrodes as a reliable platform for continuous health monitoring in demanding applications.

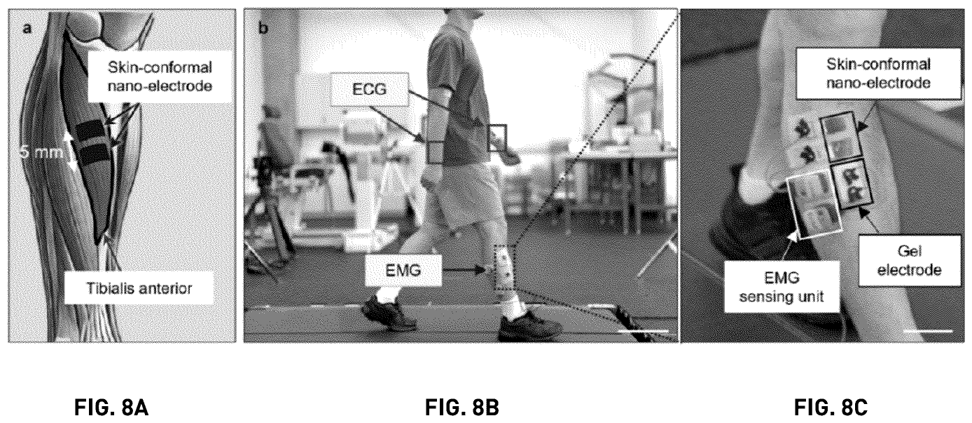

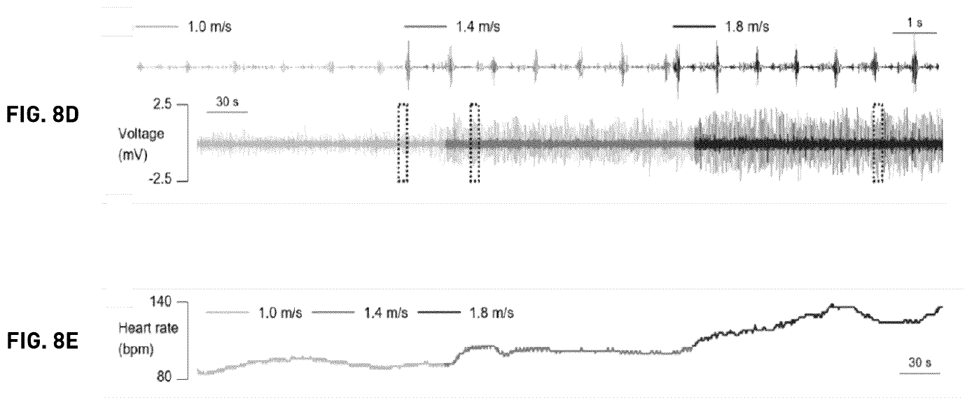

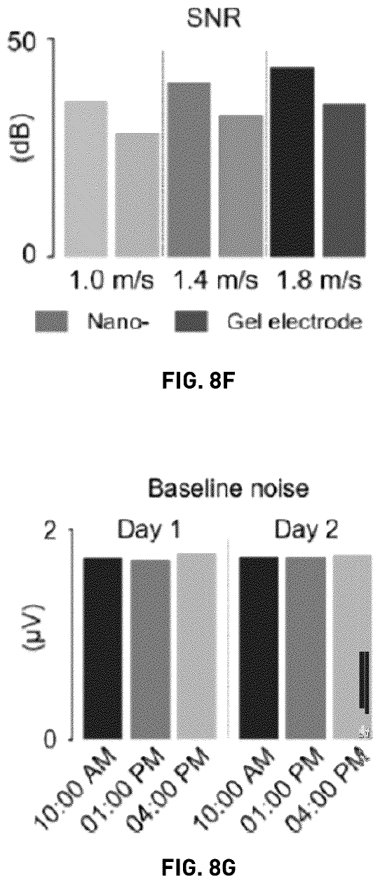

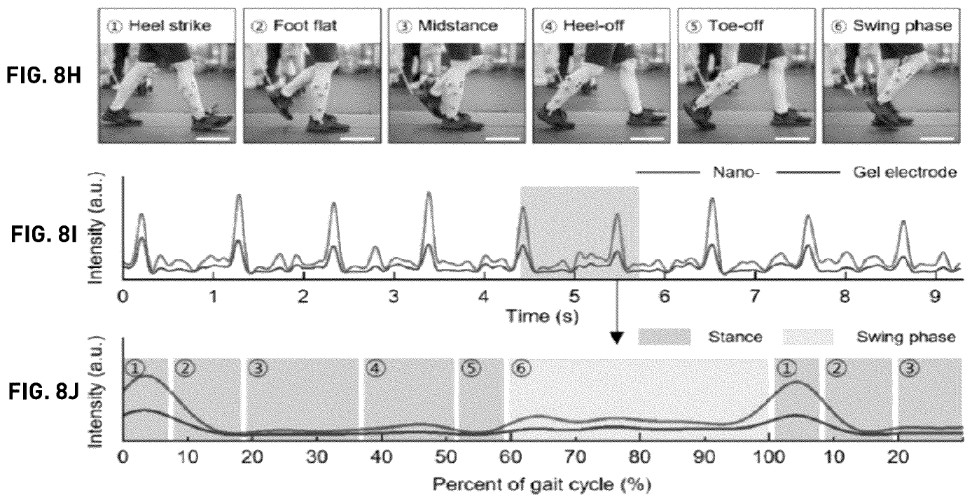

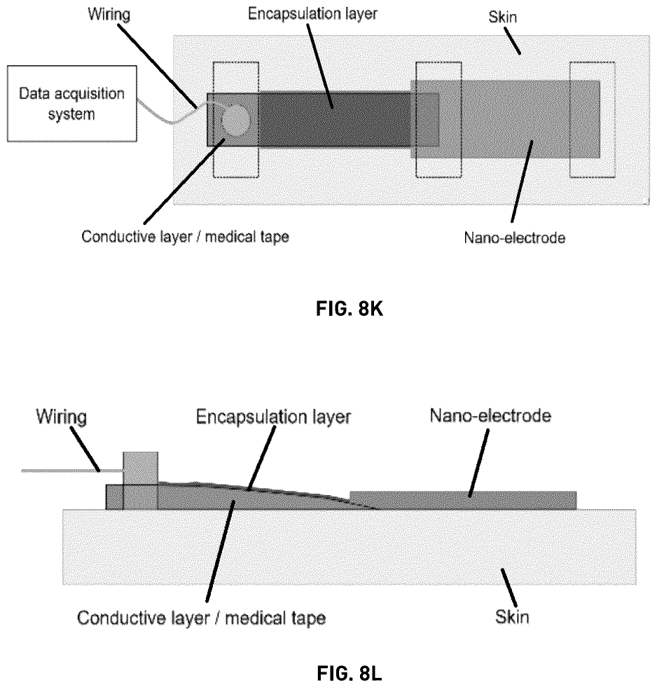

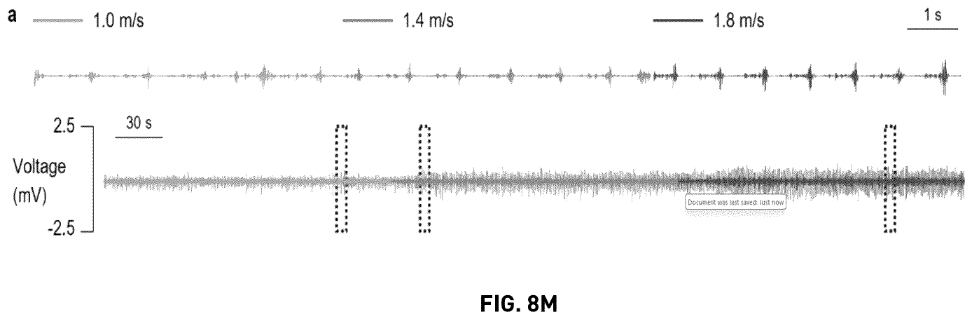

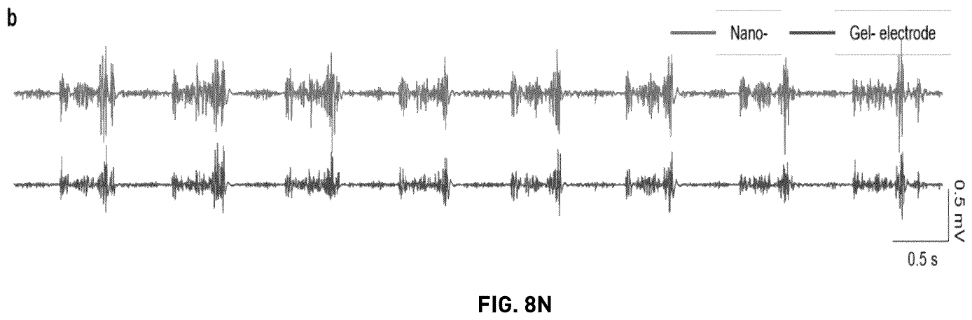

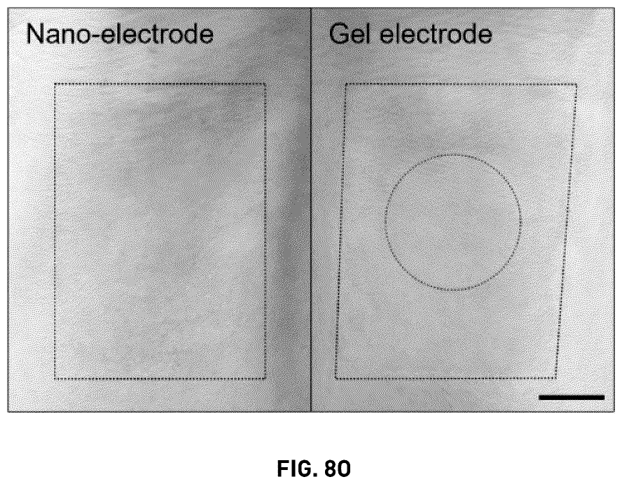

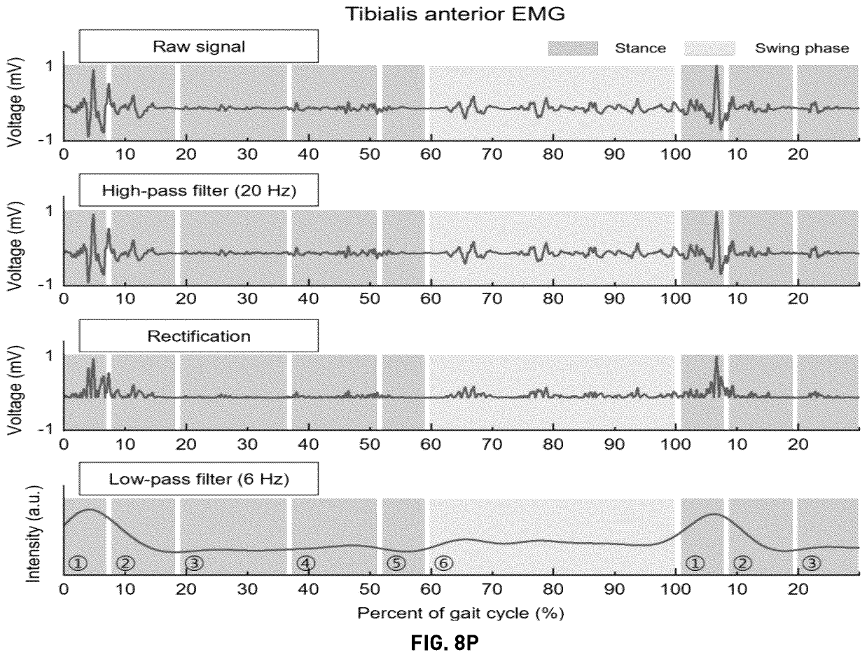

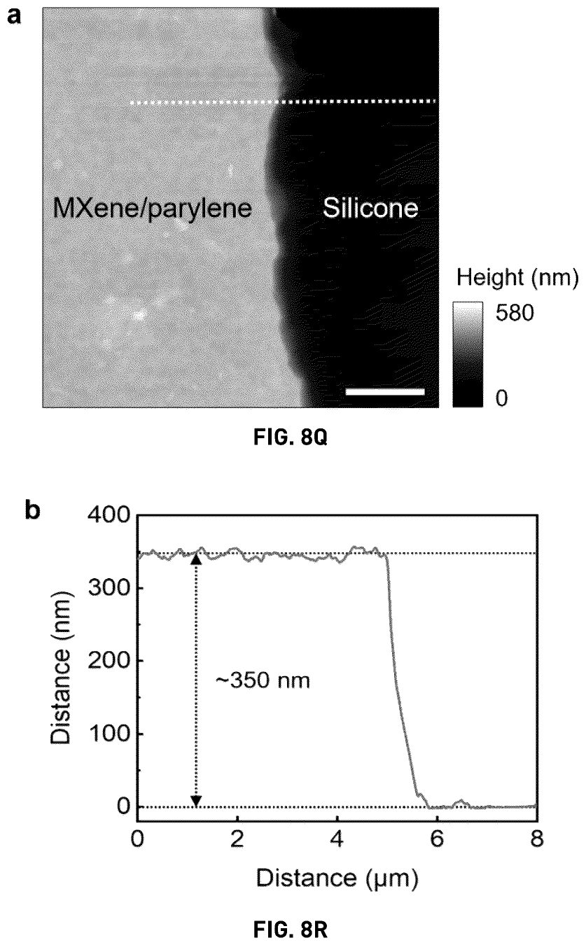

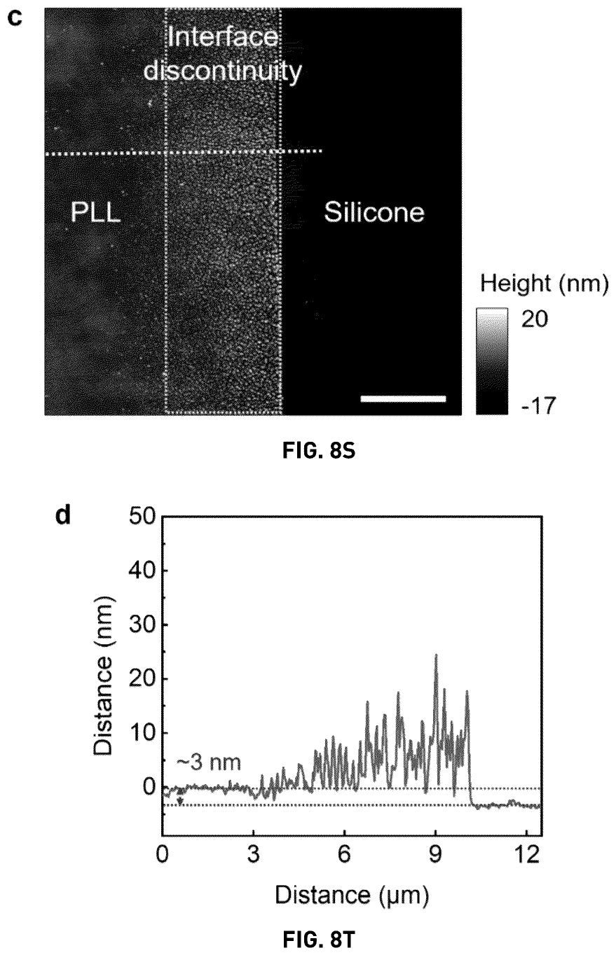

Electrophysiological signals monitoring in real-life testing environments. The tibialis anterior is one of the key muscles in the lower leg that plays a crucial role in understanding the muscle's activation and coordination during the human gait cycle. For the continuous tibialis anterior EMG monitoring, the inventors attached the skin-conformal nano-electrodes to the tibialis anterior with a 5 mm spacing between electrodes in FIG. 8A.

As shown in FIG. 8B, the inventors tested 30-year-old human gaits at constant speeds on the treadmill. To monitor the ECG signals simultaneously, the inventors attached skin-conformal nano-electrodes to the same positions as in previous experiments, and the inventors also attached gel electrodes to the tibialis anterior right below the nano-electrodes (FIG. 8B, right). In addition, wireless EMG sensing units were positioned next to the electrodes to enable real-time EMG signal monitoring. For stable wireless signal transmission and continuous EMG monitoring of the tibialis anterior muscle, a mechanical gradient design ensures secure and effortless attachment to the lower leg (FIGS. 8K and 8L).

In particular, the experiment involved attaching two sets of electrodes to the participant's right leg tibialis anterior: a pair of ultrathin electrodes and a pair of gel electrodes. After the mounted ultrathin electrodes completely dried, the participant then performed a walking test on a treadmill, maintaining speeds of 1.0 m/s (slow), 1.4 m/s (typical), and 1.8 m/s (brisk) for 3 minutes each, in consecutive order to monitor the EMG signal from the pairs of the electrodes sets. This walking protocol was repeated six times over the course of two days to analyze long-term performance of both electrodes. The participants kept wearing the electrode sets over two days to analyze their endurance during daily activities. On each day, the tests were conducted at three different times: 10:00 am, 1:00 μm, and 4:00 μm.

The inventors then increased the treadmill speed at 3-minute intervals ranging from low (1.0 m/s), to medium (1.4 m/s), and high (1.8 m/s), which covers most walking cycles (FIG. 8C). The inventors observed that as the treadmill speed increased, the magnitude of the EMG signals increased, indicating more pronounced and periodic muscle activity during gait. Peak-to-peak voltages at low, medium, and high speeds were 1.14, 2.72, and 3.25 mV, respectively, with corresponding walking intervals of 1.08, 0.97, and 0.88 s. Additionally, HR was monitored using ECG signals with the Pan-Tompkins algorithm in FIG. 8D. HR readings increased with speed: 80 bpm at low, 100-110 bpm at medium, and 120-140 bpm at high speed, showing a strong correlation between speed and heart rate. These results can demonstrate how muscle and cardiovascular responses correlate with activity intensity.

The inventors compared the tibialis anterior EMG signals of the skin-conformal nano-electrodes to those of gel electrodes. The inventors simultaneously monitored and compared the EMG of gel and nano-electrodes at three different speeds (see FIGS. 8M and 8N). In detail, at a speed of 1.0 m/s, the nano-electrodes consistently showed higher EMG signals compared to the gel electrodes (Peak-to-peak voltages: 0.81 mV). The calculated SNR of the nano-electrodes at low, medium, and high speeds was 35.6, 39.9, and 43.4 dB, respectively, which is approximately 25% higher than that of the gel electrodes, as shown in FIG. 8E. This indicates that nano-electrodes provide higher signal clarity across all speeds. Next, the inventors conducted long-term baseline noise analysis of nano-electrodes over 30 hours in FIG. 8F. The initial baseline noise was approximately 1.72 μV, and only a very minor, 1% increase in baseline noise was observed even after 30 hours, highlighting the noise stability and consistent signal quality across prolonged use without causing any visible skin irritation (see FIG. 8O).

For the in-depth analysis of muscle activity of the tibialis anterior based on the raw EMG and noise signals, the linear envelope of tibialis anterior EMG signals is essential. This technique helps in capturing the dynamic nature of human gaits, offering a clearer understanding of muscle function and its activation patterns during the walking cycles during three successive operations (see FIG. 8P). The raw signal is cleaned from irrelevant frequencies with a high-pass filter (20 Hz). The signal is then rectified with an absolute value.

Finally, high frequencies are filtered out with a low-pass filter (6 Hz). The fundamental components of a typical gait cycle are divided into two primary phases: the stance and swing phases shown in FIG. 8G. The stance phase, which constitutes about 60% of the gait cycle, involves the foot in contact with the ground and is further divided into the heel strike, foot flat, midstance, heel-off, and toe-off phases (phases 1 to 5, respectively). The swing phase (phase 6), accounting for the remaining 40%, involves the foot moving through the air and is split into the leg lift and swing forward phases. These two phases constitute the fundamental mechanics of human walking. As shown in the overlaid envelope profile (FIG. 8H), the skin-conformal nano-electrodes demonstrated higher signal intensity compared to the gel electrodes. Specifically, during the stance phase—particularly at the heel strike—the tibialis anterior exhibited a significant increase in EMG intensity as the muscle engaged to control dorsiflexion and prevent foot slapping.

The EMG signal intensity typically peaks during this motion phase. As the gait progressed into the foot flat to toe-off phases, the activity of the tibialis anterior was highly diminished, and the EMG signals dropped significantly, indicating reduced muscle engagement as the body shifted weight onto the other foot. On the other hand, during the swing phase, the tibialis anterior showed increased activity again with a lower intensity peak in the EMG signal as it lifts the foot, maintaining dorsiflexion and ensuring the toes clear the ground. Understanding the distinct stages within each phase is crucial for analyzing the muscle activity and evaluating the overall gait performance. Therefore, continuous monitoring of daily activity provides the early detection of mobility impairments, advancing the development of wearable devices for real-time health-care monitoring, diagnostics, and personalized physical therapy.

In summary, the ultrathin skin-conformal nano-electrodes of the present disclosure offer significantly enhanced physically-driven conformability and resilient electrical properties, making them a robust platform for low-motion artifact and water-resistant electrophysiological monitoring. The unique design of dual wettability, incorporating a hydrophilic MXene conductor for enhanced skin adhesion and a hydrophobic cross-linked supporting polymer layer with hydrophobic properties, ensured strong contact with the skin, even during intense motions in air and underwater. The analytical model and structural analysis demonstrated that a nano-electrode provides strong highly adherent physical conformal contact with the skin, effectively adapting to the intricate complex textures of human skin. This ability is in great contrast to conventional gel electrodes which can only achieve a certain level of conformal contact. Their adhesion weakens over time due to factors like skin movement and sweating, leading to motion artifacts, short-term usage, and skin irritation.

The ultrathin skin-conformal nano-electrodes of the present disclosure demonstrated outstanding mechanical durability and electrical stability, maintaining conformal contact and negligible resistance variation even after extensive bending/stretching cycles and days of attachment. Furthermore, the nano-electrodes exhibited low and stable skin interfacial impedance in air and underwater, ensuring reliable and high-fidelity signal collection. Hence, nano-electrodes exhibited a high to noise ratio, lower baseline noise, and, most importantly, extremely high suspension of motion artifacts under challenging environments, including those in a sauna and pool. The inventors further demonstrated the effective use of skin-conformal nano-electrodes for concurrent EMG and ECG monitoring during treadmill walking with excellent long-term stability, especially for detecting complex muscle activities.

The nano-electrodes of the present disclosure enabled high-quality electrophysiological signal acquisition with excellent long-term stability, especially for detecting tibialis anterior activity during gait cycles. The comprehensive characteristics of conformability, water resistance, and long-term stability enable reliable, high-fidelity electrophysiological skin signal acquisition, far surpassing traditional thicker electrodes across various conditions and environments. These outcomes were supported by an extensive series of structural and durability validations as well as repeatable mechanical deformation, water immersion, and sweat exposure tests. Collectively, these evaluations affirm the nano-electrodes' robust mechanical and environmental resilience, making them uniquely well-suited for sustainable, long term bioelectronic applications for continuously monitoring mobility in daily active life, offering promising wearable applications for real-time healthcare, diagnostics, personalized physical therapy, and performance monitoring.

Preparation of MXene (Ti₃C₂Tₓ) Nanosheets: Ti₃AlC₂ MAX phase was synthesized using high-purity TiC, Al, and Ti powders (Alfa Aesar, USA) combined in a 2:1:1 atomic ratio of TiC/Ti/Al. The mixture underwent ball milling for 18 hours using zirconia balls at 60 rpm, with a 2:1 ball-to-powder ratio. The milled powder was then heated in a tube furnace under flowing argon (200 cm³/min) at 1400° C. for 2 h, with 3° C./min heating and cooling rates. The resulting material was milled with a TiN-coated bit and sieved to obtain particles smaller than 38 μm. Then a 1 L reactor was employed for the etching process. A mixture of HF, water, and HCl (50:150:300 mL) was prepared, followed by gradual addition of 50 g of Ti₃AlC₂ powder over 5 minutes using a screw feeder. The mixture was stirred for 24 hours at 150 rpm while maintaining 35° C. through water cooling. After etching, the product was repeatedly washed via centrifugation at 3500 rpm until achieving neutral pH. The MXene powder was then delaminated by mixing with 1 L of deionized water and 50 g of LiCl, stirring for 24 hours at 150 rpm. Multiple washing cycles were performed until achieving a stable colloidal solution. The final solution underwent extended centrifugation to remove any remaining multilayer particles, and the stable MXene suspension was vacuum filtered through Celgard membranes (64 nm pore size, 3501 coated polypropylene) to produce free-standing films.

Fabrication of skin-conformal nano-electrodes: Glass substrates were cleaned with DI water, acetone, and isopropyl alcohol under sonication for 10 minutes each, and coated with a Micro-90 (Cole-Parmer, USA) sacrificial layer. Then, parylene films were deposited by chemical vapor deposition using a parylene coater (SCS Labcoater PDS 2010, Specialty Coating Systems, USA). Subsequently, the parylene films were treated with O2 plasma to prepare hydrophilic surfaces. A PLL solution was spin-coated (0.1 w/v % in H2O, Sigma-Aldrich, USA) at 2000 rpm for 60 s, and then an MXene solution (25 mg mL⁻¹) at 2000 rpm for 60 s, following which the system was annealed at 110° C. to remove DI water for 10 min. The as-fabricated TA MXene-based loudspeaker was then dissolved with parylene substrate on micro-90 and glass in DI water to remove the micro-90 sacrificial layer. The total thickness of the nanoelectrode and PLL layer is approximately 350 and 3 nm, respectively (see FIGS. 8Q, 8R, 8S, 8T).

Transfer process of skin-conformal nano-electrodes onto human skin: The as-fabricated skin-conformal nano-electrode on a glass substrate was placed in water. The electrode detached from the glass substrate as the Micro-90 sacrificial layer dissolved into the water. The hydrophobicity of the parylene layer and the hydrophilicity of the MXene layer caused it to float on the surface of the water, with the parylene layer facing the air and the MXene layer in contact with the water. Then, the electrode was mounted onto human skin, or commercial artificial replica skin (silicone practice fingers, DECINIEE BEAUTY CO.), by dipping the body parts, where the signal will be measured, into the water and positioning it under the film. By lifting it, the electrode was mounted on the skin, removing residual water and leaving it dry for 10 minutes before electrophysiological measurement.

Electrical characterization: The skin-electrode interfacial impedance was measured by an LCR meter system (4284A, Agilent Technologies, USA) with a range from 20 Hz to 100 kHz. The measurements were carried out by attaching 2 cm×2 cm size electrode pairs with a center-to-center distance of 5 cm on the forearm. The resistance changes during forward and backward bending motions were measured by a semiconductor analyzer (E5272A, Agilent Technologies, USA). The nano- and thick electrodes cut by 3 cm×2 cm were attached to the inner wrist and Nickel/Copper fabric conductive tapes were connected to both ends of the electrodes.

Evaluation of ECG and EMG signals of skin-conformal nano-electrodes: To investigate the real-time monitoring of ECG and EMG signals, nano- and thick electrodes were fabricated by a rectangular shape (3 cm×2 cm) and compared them with commercial Ag/AgCl gel electrodes (Red Dot, 3M Company) for their performance in the comparison of robustness against motion artifacts, stability underwater, and durability over time some. The electrodes were connected with a commercial wireless electrophysiological data acquisition device (BioRadio, Great Lakes NeuroTechnologies, USA). For ECG signal acquisition, two measurement electrodes were mounted on the wrists, with the reference electrode placed on the back of the hand. For EMG signal acquisition, two measurement electrodes were mounted on the forearm to measure the brachioradialis muscle signals during the act of grabbing various objects and motions. The collected data was wirelessly transmitted to a computer for further data analysis. The sampling rate for all signals was 2 kHz. Data analysis was performed with MATLAB software (Mathworks, USA). The SNR of electrophysiological electrodes was calculated as follows (see also discussion above regarding use of ‘pwelch’ command):

$$SNR = 20 log ( \frac{{V}_{rms , signal}}{{V}_{rms , noise}} ) \qquad ( 19 )$$

where V_{rms,signal} is the RMS value of the signal (frequency range: 0.5-100 Hz) and V_{rns,noise} is the RMS value of the noise (frequency range: >100 Hz).

For EMG signal acquisition while walking on the treadmill, Another wireless electrophysiological data acquisition device (Ultium EMG, Noraxon, USA) was used to attach the acquisition system on the calf securely. The skin-conformal nano-electrodes and commercial gel (Dual EMG electrodes, Noraxon, USA) electrodes were attached to the subject's tibialis anterior muscle. All ECG data were filtered by a high-pass filter (0.5 Hz, Butterworth, 3rd) and all EMG data were filtered by a high-pass filter (10 Hz, Butterworth, 3rd). The signal qualities were quantified with RMS and SNR signals.

Characterization: Contact angles of MXene and parylene surface were characterized with an optical contact angle meter (KSV CAM 101) and a CCD camera (DMK 23U618, Imaging Source). Optical images were obtained using a digital optical microscope (10″ HDMI LCD Digital Microscope 1500×, Dcorn, USA), characterized the morphology of the skin-conformal nanoelectrodes by field emission SEM (SU-8230, Hitachi, Japan), and determined the thickness of the MXene conductor by atomic force microscopy (Dimension Icon AFM, Bruker, USA). A profilometry height profile of skin-conformal nano-electrodes on the artificial skin was characterized by an optical surface profiler (VK-X3000, Keyence, USA).

The optical transmittance of the skin-conformal nano-electrodes was measured using a UV-vis spectrophotometer (UV-3600 Plus, Shimadzu, Japan). The zeta potential of the MXene and PLL was analyzed by a Zetasizer device (Nano ZS, Malvern, UK). All measurements were repeated on at least five independently fabricated samples for each condition to ensure reproducibility.

The nano-electrode of the present disclosure can be applied to such applications as wearable healthcare diagnostics and continuous health monitoring; medical devices for real-time tracking of vital signals, such as ECG and EMG, supporting the diagnosis and management of cardiac, neurological, and muscular disorders. Its flexibility and water resistance make it suitable for fitness tracking, rehabilitation, human-machine interfaces, and aquatic environments, enabling accurate monitoring during physical activities, virtual reality interactions, and underwater use. Additionally, the nano-electrodes can enhance bipotential accuracy in consumer electronics like smartwatches and fitness bands.

The nano-electrode of the present disclosure has several advantages over existing technologies, such as, (a) its ultrathin (around 300 nm) skin-like design ensures more secure and conformal contact with the skin, reducing air gaps and enhancing signal quality; (b) the high conformability also minimizes motion artifacts, providing more stable and accurate monitoring during physical activities compared to traditional gel or dry electrodes; (c) the nano-electrodes are water resistant, maintaining reliable performance even in wet conditions, which is not possible with conventional electrodes; (d) the dual-layer structure, featuring hydrophilic MXene and hydrophobic parylene, improves 3 times increase in the signal-to-noise (SNR) and reduces baseline noise by 22%, resulting in high-fidelity electrophysiological signals; (e) the nano-electrodes demonstrate long-term stability, maintaining consistent performance over extended periods, unlike gel electrodes that degrade and require frequent replacement; and (f) the nano-electrodes provide greater comfort and safety, as its flexible, thin design minimizes skin irritation, making it more comfortable for users.

The nano-electrode of the present disclosure can be configured for long-term performance under extreme conditions, such as high temperatures, heavy sweating, or prolonged submersion. The nano-electrode can be connectable to a data-collecting device for continuous monitoring, such as for wireless applications.

In accordance with aspects of the present disclosure, and with reference to FIG. 1, a schematic showing a method 100 of fabricating a nano-electrode is presented. In some embodiments, the nano-electrode is a skin-conformal nano-electrode. In some embodiments, the method 100 includes coating 110 a glass substrate with a sacrificial layer, depositing 120 a parylene on the sacrificial layer, coating 150 the parylene with an MXene solution, annealing a combination of the sacrificial layer, the parylene and the MXene solution to form the nano-electrode, and removing 160 the sacrificial layer. In some embodiments, the MXene solution is an MXene (Ti₃C₂Tₓ) nanosheet. In some embodiments, the method 100 further includes applying or transferring 170 the nano-electrode onto skin or a skin surface.

In some embodiments, the method 100 includes coating 130 the parylene with a poly-L-lysine (PLL) solution. In some embodiments, the coating 130 of the parylene with the poly-L-lysine (PLL) solution is performed by spin-coating. In some embodiments, the spin-coating is performed at about 2000 rpm for a period of about 60 seconds. In some embodiments, the annealing 160 of the combination of the sacrificial layer, parylene and the MXene is performed at a temperature of about 110° C. for a period of about 10 minutes. In some embodiments, the nano-electrode formed has a thickness of about 350 nm. In some embodiments, the PLL layer formed has a thickness of about 2 nm to 4 nm. In some embodiments, the PLL layer formed has a thickness of about 3 nm.

In some embodiments, the parylene is a parylene film, and the depositing parylene on the sacrificial layer includes chemical vapor deposition (CVD) of a parylene film onto the sacrificial layer. In some embodiments, the depositing 120 parylene on the sacrificial layer includes treating 140 the parylene film with an O₂ plasma to prepare the parylene as a hydrophilic surface. In some embodiments, the sacrificial layer includes an aqueous solution including one or more of chelating agents, builders, a solubilizer, and a mixture of anionic and non-ionic surfactants. In some embodiments, the sacrificial layer includes a Cole-Parmer® Micro-90® solution.

In some embodiments, the nano-electrode formed using the above method 100, or any other method described herein, may be transferred and attached to a skin surface. In some embodiments, the nano-electrode formed on the glass substrate is placed in water. In some embodiments the nano-electrode detaches from the glass substrate as the sacrificial layer dissolves in the water. The hydrophobicity of the parylene layer and the hydrophilicity of the MXene layer causes the nano-electrode to float in the water, with the parylene layer facing upwards toward the air, and the MXene layer facing downwards in contact with the water. In some embodiments, the transfer further includes dipping the skin surface in the water and positioning the skin surface below the MXene layer of the nano-electrode. In some embodiments, the skin surface is then lifted towards MXene layer to mount onto the skin surface. In some embodiments, the nano-electrode and the skin surface is left to dry for about 10 minutes.

In some embodiments, a method of applying a nano-electrode to a skin surface includes floating the nano-electrode in water, submerging the skin surface in the water under the nano-electrode, stretching the skin surface, lifting the skin surface being stretched until the nano-electrode mounts to the skin surface, releasing the skin surface, and drying the nano-electrode mounted on the skin surface.

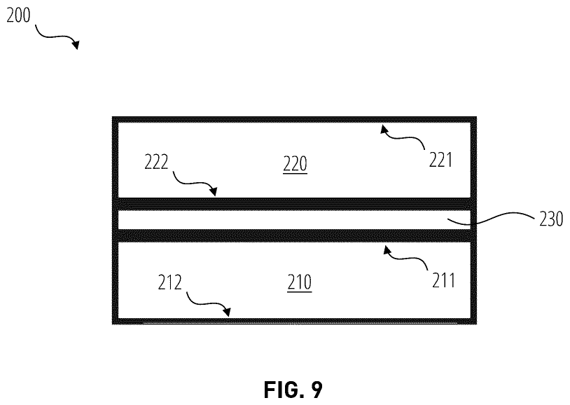

In accordance with aspects of the present disclosure, and with reference to FIG. 9, a nano-electrode 200 includes a hydrophilic conductive layer 210 and a hydrophobic non-conductive layer 220. In some embodiments, the hydrophilic conductive layer 210 includes a first side surface 211 and a second side surface 212 opposite from the first side surface 211. In some embodiments, the first side surface 211 is a top surface, and the second side surface 212 is a bottom surface. In some embodiments, the hydrophobic non-conductive layer 220 includes a first side surface 221 and a second side surface 222 opposite from the first side surface 221. In some embodiments, the first side surface 221 is a top surface, and the second side surface 222 is a bottom surface.

In some embodiments, the hydrophobic non-conductive layer 220 is configured to cover the first side surface 211 of the hydrophilic conductive layer 210. In some embodiments, the second side surface 212 of the hydrophilic conductive layer 210 is configured to contact skin or a skin surface. In some embodiments, the second side surface 212 of the hydrophilic conductive layer 210 is configured to adhere to the skin or the skin surface. In some embodiments, an outer perimeter of the hydrophobic non-conductive layer 220 extends beyond an outer perimeter of the hydrophilic conductive layer 210. In other words, an overall footprint of the hydrophobic non-conductive layer 220 is larger than an overall footprint of the hydrophilic conductive layer 210 such that the hydrophobic non-conductive layer 220 completely covers over the hydrophilic conductive layer 210. In some embodiments, the hydrophilic conductive layer 210 has a contact angle of water of about 88 degrees. In some embodiments, the hydrophobic non-conductive layer 220 has a contact angle of water of about 34 degrees.

In some embodiments, a thickness of the hydrophobic non-conductive layer 220 is about 200 nm to 400 nm. In some embodiments, a thickness of the hydrophobic non-conductive layer 220 is about 250 nm to 350 nm. In some embodiments, a thickness of the hydrophobic non-conductive layer 220 is about 275 nm to 325 nm. In some embodiments, a thickness of the hydrophobic non-conductive layer 220 is about 300 nm. In some embodiments, the hydrophobic non-conductive layer 220 includes parylene. In some embodiments, the hydrophobic non-conductive layer 220 includes crosslinked chemical vapor deposition (CVD) fabricated parylene nanofilm.

In some embodiments, a thickness of the hydrophilic conductive layer 210 is about 40 nm to 60 nm. In some embodiments, a thickness of the hydrophilic conductive layer 210 is about 45 nm to 55 nm. In some embodiments, a thickness of the hydrophilic conductive layer 210 is about 50 nm. In some embodiments, the hydrophilic conductive layer 210 includes at least one of an MXene, an MXene layer, an MXene sheet, an MXene nanosheet, and an MXene film. In some embodiments, the hydrophilic conductive layer 210 includes a conductive layer of a 2D MXene phase (Ti₃C₂Tₓ).

In some embodiments, the nano-electrode 200 further comprises an adhesive layer 230 configured to attach the hydrophilic conductive layer 210 and the hydrophobic non-conductive layer 220. In some embodiments, the adhesive layer 230 contacts the first side surface 211 of the hydrophilic conductive layer 210 and the adhesive layer 230 contacts the second side surface 222 of the and the hydrophobic non-conductive layer 220. In some embodiments, the adhesive layer 230 includes poly-L-lysine (PLL).

In some embodiments, the hydrophilic conductive layer 210 of the nano-electrode 220 is operable to sense one or more of an electrocardiogram (ECG) signal, an electromyogram (EMG) signal, an electrooculogram signal, and an electroencephalogram signal.

In some embodiments, a nano-electrode 220 includes a hydrophilic layer with a MXene material and a hydrophobic layer with parylene. The hydrophilic layer has a first side surface and a second side surface opposite the first side surface, and the hydrophobic layer covers the first side surface of the hydrophilic layer.

In some embodiments, the second side surface of the hydrophilic layer is configured to self-adhere to the skin surface. In some embodiments, a thickness of the hydrophobic layer is about 250 nm to 350 nm. In some embodiments, a thickness of the hydrophobic layer is about 300 nm. In some embodiments, a thickness of the hydrophilic layer is about 40 nm to 60 nm. In some embodiments, a thickness of the hydrophilic layer is about 50 nm.

Additionally, where a method described above or a method claim below does not explicitly require an order to be followed by its steps or an order is otherwise not required based on the description or claim language, it is not intended that any particular order be inferred. Likewise, where a method claim below does not explicitly recite a step mentioned in the description above, it should not be assumed that the step is required by the claim.

It is noted that the description and claims may use geometric or relational terms, such as right, left, above, below, upper, lower, top, bottom, linear, arcuate, elongated, parallel, perpendicular, etc. These terms are not intended to limit the disclosure and, in general, are used for convenience to facilitate the description based on the examples shown in the figures. In addition, the geometric or relational terms may not be exact. For instance, walls may not be exactly perpendicular or parallel to one another because of, for example, roughness of surfaces, tolerances allowed in manufacturing, etc., but may still be considered to be perpendicular or parallel.

It will also be appreciated by those skilled in the art that modifications can be made to the example embodiments described herein without departing from the invention. Structural features of systems and apparatuses described herein can be replaced with functionally equivalent parts or omitted entirely. Moreover, it will be appreciated that features from the embodiments can be combined with each other without departing from the disclosure.

What is claimed is:

1. A nano-electrode, comprising:
a hydrophilic conductive layer having a first side surface and a second side surface opposite of the first side surface of the hydrophilic conductive layer; and
a hydrophobic non-conductive layer having a first side surface and a second side surface opposite of the first side surface of the hydrophobic non-conductive layer, and the hydrophobic non-conductive layer is configured to cover the first side surface of the hydrophilic conductive layer,
wherein the second side surface of the hydrophilic conductive layer is configured to attach to a skin surface.

2. The nano-electrode of claim 1, wherein a thickness of the hydrophobic non-conductive layer is about 250 nm to 350 nm.

3. The nano-electrode of claim 1, wherein a thickness of the hydrophobic non-conductive layer is about 300 nm.

4. The nano-electrode of claim 1, wherein the hydrophobic non-conductive layer comprises parylene.

5. The nano-electrode of claim 4, wherein the parylene is a crosslinked chemical vapor deposition (CVD) fabricated parylene nanofilm.

6. The nano-electrode of claim 1, wherein a thickness of the hydrophilic conductive layer is about 40 nm to 60 nm.

7. The nano-electrode of claim 1, wherein a thickness of the hydrophilic conductive layer is about 50 nm.

8. The nano-electrode of claim 1, wherein the hydrophilic conductive layer comprises a MXene (Ti₃C₂Tₓ) nanosheet.

9. The nano-electrode of claim 1, further comprising an adhesive layer between the hydrophilic conductive layer and the hydrophobic non-conductive layer.

10. The nano-electrode of claim 9, wherein the adhesive layer comprises poly-L-lysine (PLL).

11. The nano-electrode of claim 9, wherein the adhesive layer contacts the first side surface of the hydrophilic conductive layer and contacts the second side surface of the hydrophobic non-conductive layer.

12. A nano-electrode for attachment to a skin surface to sense electrophysiological signals, comprising:
a hydrophilic layer with an MXene material, the hydrophilic layer having a first side surface and a second side surface opposite the first side surface; and
a hydrophobic layer comprising parylene, the hydrophobic layer covering the first side surface of the hydrophilic layer.

13. The nano-electrode of claim 12, wherein the second side surface of the hydrophilic layer is configured to self-adhere to the skin surface.

14. The nano-electrode of claim 12, wherein a thickness of the hydrophobic layer is about 250 nm to 350 nm.

15. The nano-electrode of claim 12, wherein a thickness of the hydrophilic layer is about 40 nm to 60 nm.

16. A method of applying the nano-electrode of claim 12 to a skin surface, the method comprising:
floating the nano-electrode in water;
submerging the skin surface in the water under the nano-electrode;
stretching the skin surface;
lifting the skin surface being stretched until the nano-electrode mounts to the skin surface; and
releasing the skin surface.

17. A method of fabricating a nano-electrode, comprising:
coating a glass substrate with a sacrificial layer;
depositing a parylene on the sacrificial layer;
coating the parylene with an MXene solution;
annealing a combination of the sacrificial layer, the parylene and the MXene solution; and
removing the sacrificial layer.

18. The method of claim 17, further comprising coating the parylene with a poly-L-lysine (PLL) solution prior to coating the parylene with the MXene solution.

19. The method of claim 17, further comprising treating the parylene with O₂ plasma to prepare the parylene as a hydrophilic surface.

20. The method of claim 17, wherein the depositing the parylene on the sacrificial layer includes chemical vapor deposition (CVD) of a parylene film onto the sacrificial layer.
